(19) United States
(12) Patent Application Publication
(10) Pub. No.: US 2008/0161344 A1
(43) Pub. Date: Jul. 3, 2008

(54) Melanin Concentrating Hormone Receptor Ligands: Substituted Tetrahydroisoquinoline Analogues

(76) Inventors: Cheryl K. Steenstra, Bath, ME (US); Taeyoung Yoon, Newton, MA (US); John M. Peterson, Durham, CT (US)

Correspondence Address: NEUROGEN CORPORATION, 35 NORTHEAST INDUSTRIAL ROAD, BRANFORD, CT, 06405, US

(21) Appl. No.: 10/597,641

(22) PCT Filed: Feb. 9, 2005
(86) PCT No.: PCT/US2005/004107

§ 371 Date: Aug. 2, 2006

Related U.S. Application Data
(60) Provisional application No. 60/543,117, filed on Feb. 9, 2004.

(51) Int. Cl. A61K 31/472; C07D 217/20; C07D 401/02

(52) U.S. Cl. 514/307; 546/149

(57) ABSTRACT

Melanin concentrating hormone receptor ligands (especially substituted tetrahydroisoquinoline analogues), capable of modulating MCH receptor activity, are provided. Such ligands may be used to modulate MCH binding to MCH receptors in vivo or in vitro, and are particularly useful in the treatment of a variety of metabolic, feeding and sexual disorders in humans, domesticated companion animals and livestock animals. Pharmaceutical compositions and methods for treating such disorders are provided, as are methods for using such ligands for detecting MCH receptors (e.g., receptor localization studies).

### FIELD OF THE INVENTION

This invention relates generally to substituted tetrahydroisoquinoline analogues that are melanin concentrating hormone receptor modulators. The invention further relates to the use of such compounds for treating a variety of metabolic, eating and sexual disorders, and as probes for the detection and localization of MCH receptors.

### BACKGROUND OF THE INVENTION

Melanin concentrating hormone, or MCH, is a cyclic 19 amino acid neuropeptide first identified as a regulator of skin coloration in fish and other vertebrates, and subsequently as a regulator of food intake and energy balance in higher vertebrates. In many species, including humans, MCH is produced in the hypothalamus. MCH is also produced at various peripheral sites, including the gastrointestinal tract and testis.

The postulated role of MCH in feeding behavior and body weight regulation is confirmed by the finding that i.c.v. injection of MCH increases caloric consumption in rats over similarly treated control animals. Furthermore, rats having the ob/ob genotype exhibit a 50-80% increase in MCH mRNA expression as compared to leaner ob/+ genotype mice, and prepro-MCH knockout mice, as well as MCH receptor knockout mice, are leaner than normal mice, due to hypophagia and an increased metabolic rate.

MCH activity is mediated via binding to specific receptors. Like other G protein-coupled receptors (e.g., neuropeptide Y (NPY) and beta-adrenergic receptors), MCH receptors are membrane-spanning proteins, generally found on cell surfaces, that consist of a single contiguous amino acid chain comprising an extracellular N-terminal domain, seven membrane-spanning alpha helical domains (connected by three intracellular loop domains alternating with three extracellular loop domains), and an intracellular C-terminal domain. Signal transduction is typically initiated by the binding of extracellular MCH to the receptor. This elicits conformational changes in the extracellular domains. When the receptor is functioning properly, these conformational changes propagate through the transmembrane domains and result in a coordinated change in the intracellular portions of the receptor. This precise alteration in the intracellular domains acts to trigger the associated G-protein complex to modulate intracellular signaling.

Human Melanin Concentrating Hormone Receptor-1 (MCH1R) is a 353 amino acid, 7-transmembrane, alpha-helical, G protein-coupled receptor, initially reported as orphan receptor SLC-1. Immunohistochemistry studies of rat brain sections indicate that MCH1R is widely expressed in brain, MCH1R expression is found in olfactory tubercle, cerebral cortex, substantia nigra, basal forebrain CA1, CA2, and CA3 fields of the hippocampus, amygdala, and in nuclei of the hypothalamus, thalamus, midbrain and hindbrain. Strong signals are observed in the ventromedial and dorsomedial nuclei of the hypothalamus, two areas of the brain involved in feeding behavior. Upon binding MCH, MCH1R recombinantly expressed in HEK 293 cells mediates a dose dependent release of intracellular calcium. Cells expressing MCH1R also exhibit a pertussis toxin sensitive dose-dependent inhibition of forskolin-elevated cyclic AMP, indicating that the receptor couples to a G_{i/o}, G-protein alpha subunit. Certain monkey and human MCH1R sequences, as well as various chimeric MCH1R proteins, have been disclosed in U.S. patent application Ser. No. 10/309,515 (published as 2003/0114644 on Jun. 19, 2003).

Recently, a second MCH receptor (designated MCH2R) was identified. MCH2R has an overall amino acid identity of more than 30% with MCH1R, and is detected specifically in the same regions of the brain as MCH1R. Monkey and canine MCH2R sequences, as well as various chimeric MCH2R proteins, have been disclosed in U.S. patent application Ser. No. 10/291,990 (published as 2003/0148457 on Aug. 7, 2003).

Agents capable of modulating MCH receptor activity are highly desirable for the treatment of a variety of diseases and disorders, including obesity, eating disorders (e.g., bulimia and anorexia), sexual disorders (e.g., anorgasmic or psychogenic impotence) and metabolic disorders, such as diabetes. Small molecule, non-peptide antagonists of MCH receptors would be of particular value for such therapies.

### SUMMARY OF THE INVENTION

The present invention provides compounds of Formula I, and pharmaceutically acceptable salts thereof:

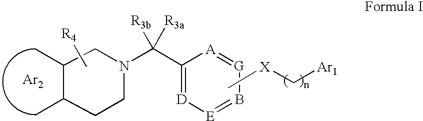

### Within Formula I:

A, E, and D are independently CR₂ or N; one of B and G is chosen from CR₂ and N; and the other of B and G is a carbon atom covalently bound to the group

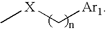

X is O, NH or CH₂
n is 0 or 1.
Ar₁ is phenyl or a 6-membered aromatic heterocycle, each of which is substituted with from 0 to 4 substituents independently chosen from Rₐ; or two adjacent substituents are taken together to form, with the ring atoms to which they are bound, a fused 5- or 6-membered ring substituted with from 0 to 4 substituents independently chosen from Rₐ.

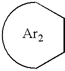

represents fused phenyl or a fused 6-membered aromatic heterocycle, each of which is substituted with from 0 to 4 substituents independently chosen from Rₐ; or two adjacent substituents are taken together to form, with the ring atoms to which they are bound, a fused 5- or 6-membered ring substituted with from 0 to 3 substituents independently chosen from Rₐ.
R₂ is independently chosen at each occurrence from hydrogen, hydroxy, halogen, amino, nitro, cyano, C₁-C₆alkyl, C₂-C₆alkenyl, C₂-C₆alkynyl, C₁-C₆alkoxy, C₃-C₇cycloalkyl(C₀-C₄alkyl), C₁-C₆haloalkyl, C₁-C₆haloalkoxy, and mono- and di-(C₁-C₄alkyl)amino.
R₃ₐ and R_{3b} are independently hydrogen, hydroxy, halogen, cyano, C₁-C₆alkyl, C₂-C₆alkenyl, C₂-C₆alkynyl, C₁-C₆alkoxy, C₃-C₇cycloalkyl(C₀-C₄alkyl), C₁-C₆haloalkyl or C₁-C₆haloalkoxy; or R₃ₐ and R_{3b} are taken together to form an oxo group.
R₄ represents from 0 to 3 substituents independently chosen from hydroxy, halogen, amino, nitro, cyano, C₁-C₆alkyl, C₂-C₆alkenyl, C₂-C₆alkynyl, C₁-C₆alkoxy, C₃-C₇cycloalkyl(C₀-C₄alkyl), C₁-C₆haloalkyl and C₁-C₆haloalkoxy.
Rₐ is independently chosen at each occurrence from:
(i) hydroxy, halogen, amino, aminocarbonyl, cyano, nitro and —COOH; and
(ii) C₁-C₈alkyl, C₂-C₈alkenyl, C₂-C₈alkynyl, C₁-C₈alkoxy, C₃-C₇cycloalkyl(C₀-C₄alkyl), C₁-C₈haloalkyl, C₁-C₈haloalkoxy, C₁-C₈alkanoyl, C₃-C₈alkanone, C₂-C₈alkoxycarbonyl, C₂-C₈alkanoyloxy, C₁-C₈alkylthio, C₂-C₈alkyl ether, phenylC₀-C₄alkyl, phenylC₀-C₄alkoxy, mono- and di-(C₁-C₆alkyl)aminoC₀-C₆alkyl, and (4- to 7-membered heterocycle)C₀-C₄alkyl; each of which is substituted with from 0 to 3 substituents independently chosen from hydroxy, halogen, amino, cyano, C₁-C₄alkyl, C₁-C₄alkoxy, C₁-C₄haloalkyl, C₁-C₄haloalkoxy and mono- and di-(C₁-C₄alkyl)amino.

Within certain embodiments, if (i)

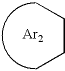

is unsubstituted phenyl, di-methoxy phenyl or phenyl substituted with phenyl (C₁-C₂alkoxy); (ii) A, B, E, and D are each CR₂; (iii) G is a carbon atom covalently bound to

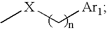

and (iv) Ar₁ phenyl, then Ar₁ is substituted at the position para to the point of attachment with a substituent other than halogen. Within further embodiments, if (i)

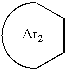

is unsubstituted phenyl, di-methoxy phenyl, or 3-methoxy-4-benzyloxyphenyl, R₃ₐ and R_{3b} are both hydrogen, and R₄ represents 0 substituents; (ii) A, B, G, and D are each CR₂; and (iii) E is a carbon atom covalently bound to

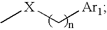

and (iv) Ar₁ is phenyl, then Ar₁ is substituted with at least one additional substituent that is not methyl, methoxy or ethoxy.

Within certain aspects, compounds as described herein are MCH receptor modulators and exhibit a Kᵢ; of no greater than 1 micromolar, 500 nanomolar, 100 nanomolar, or 10 nanomolar in a MCH receptor binding assay and/or have an EC₅₀ or IC₅₀ value of no greater than 1 micromolar, 500 nanomolar, 100 nanomolar, or 10 nanomolar in an assay for determining MCH receptor agonist or antagonist activity.

Within certain aspects, compounds or salts as described herein are labeled with a detectable marker (e.g., radiolabeled or fluorescein conjugated).

The present invention further provides, within other aspects, pharmaceutical compositions comprising at least one compound or salts as described herein (i.e., a compound Formula I or a pharmaceutically acceptable salt thereof) in combination with a physiologically acceptable carrier or excipient. Within certain embodiments, a pharmaceutical composition provided herein may further comprise one or more additional active agents (i.e., drugs). Pharmaceutical compositions provided herein may be formulated, for example, as an injectable fluid, an aerosol, a cream, a gel, a pill, a capsule, a syrup, or a transdermal patch.

The present invention further provides, within other aspects, methods for treating a disease or disorder associated with MCH receptor activation, comprising administering to a patient in need of such treatment a therapeutically effective amount of a MCH receptor modulator as described above. Such diseases and disorders include, for example, eating disorders (e.g., obesity and bulimia nervosa), sexual disorders, diabetes, heart disease, and stroke. The MCH receptor modulator may be administered orally, or via another means such as intranasally, intravenously, or topically. Within certain embodiments, the patient is a human, companion animal, or livestock animal.

Methods are provided, within other aspects, for determining the presence or absence of MCH receptor in a sample, comprising: contacting a sample with a compound as described above under conditions that permit binding of the compound to MCH receptor; and detecting a level of the compound bound to MCH receptor. Within certain embodiments, the compound is radiolabeled, and the step of detection comprises: separating unbound compound from bound compound; and determining an amount of bound compound in the sample. Detection may be achieved, for example, using autoradiography.

The present invention further provides, within other aspects, methods for modulating binding of ligand to MCH receptor. Certain such methods are performed in vitro, and comprise contacting MCH receptor with MCH receptor modulator, as described above under conditions and in an amount sufficient to detectably modulate MCH binding to MCH receptor. Other such methods may be performed in vivo, and comprise contacting cells expressing MCH receptor with a compound or modulator as described above in an amount sufficient to detectably modulate MCH binding to cells expressing a cloned MCH receptor in vitro. Modulation of MCH binding may be determined, for example, using a ligand binding assay as provided herein.

Methods are further provided for modulating binding of MCH to MCH receptor in a patient, comprising administering to a patient (i.e., a human or non-human animal) a compound or modulator as described above. Patients include, for example, companion animals such as dogs.

Within certain embodiments of the above methods, the modulation is inhibition and/or the MCH receptor is a human MCH receptor, such as human MCH1R or MCH2R, or is a chimeric MCH1R or MCH2R receptor in which one or more domains are replaced with a corresponding domain of a different G protein-coupled receptor.

Within further aspects, the present invention provides methods for modulating the signal-transducing activity of MCH receptor, comprising contacting an MCH receptor, either in vivo or in vitro, with an amount of an MCH receptor modulator sufficient to detectably alter MCH receptor activity, under conditions suitable for binding of MCH to MCH receptor. Preferably, the MCH receptor is a MCH1R.

Packaged pharmaceutical preparations, comprising: (a) a pharmaceutical composition as described above in a container; and (b) instructions for using the composition to treat a patient suffering from a disease or disorder associated with MCH receptor activation. Such disorders include, for example eating disorders (e.g., obesity and bulimia nervosa), sexual disorders, diabetes, heart disease, and stroke, are also provided herein.

In yet another aspect, methods of preparing the compounds disclosed herein, including the intermediates, are also provided herein.

These and other aspects of the present invention will become apparent upon reference to the following detailed description.

### DETAILED DESCRIPTION OF THE INVENTION

As noted above, the present invention provides compounds of Formula I. Certain preferred compounds are MCH receptor modulators that may be used in vitro or in vivo, to inhibit MCH binding to MCH receptors, activate MCH receptors, or to otherwise modulate MCH receptor activity in a variety of contexts, as discussed in further detail below.

### Terminology

Compounds are generally described herein using standard nomenclature. For compounds having asymmetric centers, it should be understood that (unless otherwise specified) all of the optical isomers and mixtures thereof are encompassed. In addition, compounds with carbon-carbon double bonds may occur in Z- and E-forms, with all isomeric forms of the compounds being included unless otherwise specified. Where a compound exists in various tautomeric forms, a recited compound is not limited to any one specific tautomer, but rather is intended to encompass all tautomeric forms. Compound descriptions are intended to encompass compounds with all possible isotopes of atoms occurring in the compounds. Isotopes are those atoms having the same atomic number but different mass numbers. By way of general example, and without limitation, isotopes of hydrogen include tritium and deuterium and isotopes of carbon include ¹¹C, 13C and ¹⁴C. Certain compounds are described herein using a general formula that includes variables (e.g., R₂, Ar₁, Ar₂). Unless otherwise specified, each variable within such a formula is defined independently of any other variable, and any variable that occurs more than one time in a formula is defined independently at each occurrence. In general, the variables (e.g., R₁, R₂, Ar) may have any definition described herein that results in a stable compound.

A “pharmaceutically acceptable salt” of a compound recited herein is an acid or base salt that is suitable for use in contact with the tissues of human beings or animals without excessive toxicity carcinogenicity, and preferably without irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)ₙ—COOH where n is 0-4, and the like. Similarly, pharmaceutically acceptable cations include, but are not limited to sodium, potassium, calcium, aluminum, lithium, and ammonium. Those of ordinary skill in the art will recognize further pharmaceutically acceptable salts for the compounds provided herein, including those listed by Remington's Pharmaceutical Sciences, 17th ed., Mack Publishing Company, Easton, Pa., p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in water or in an organic solvent, or in a mixture of the two; generally, the use of nonaqueous media, such as ether, ethyl acetate, ethanol, isopropanol, or acetonitrile, is preferred.

It will be apparent that each compound of Formula I may, but need not, be formulated as a hydrate, solvate or non-covalent complex. In addition, the various crystal forms and polymorphs are within the scope of the present invention. Also provided herein are prodrugs of the compounds of Formula I. A “prodrug” is a compound that may not fully satisfy the structural requirements of the compounds provided herein, but is modified in vivo, following administration to a patient, to produce a compound of Formula I. For example, a prodrug may be an acylated derivative of a compound as provided herein. Prodrugs include compounds wherein hydroxy, amine or sulfhydryl groups are bonded to any group that, when administered to a mammalian subject, cleaves to form a free hydroxyl, amino or sulfhydryl group, respectively. Examples of prodrugs include, but are not limited to, acetate, formate, and benzoate derivatives of alcohol and amine functional groups within the compounds provided herein. Prodrugs of the compounds provided herein may be prepared by modifying functional groups present in the compounds in such a way that the modifications are cleaved in vivo to yield the parent compounds.

As used herein, the term “alkyl” refers to a straight chain or branched chain saturated aliphatic hydrocarbon. An alkyl group may be bonded to an atom within a molecule of interest via any chemically suitable portion. Alkyl groups include groups having from 1 to 8 carbon atoms (C₁-C₈alkyl), from 1 to 6 carbon atoms (C₁-C₆alkyl) and from 1 to 4 carbon atoms (C₁-C₄alkyl), such as methyl, ethyl, propyl, isopropyl, n-butyl, sec-butyl, tert-butyl, pentyl, 2-pentyl, isopentyl, neopentyl, hexyl, 2-hexyl, 3-hexyl, 3-methylpentyl, cyclopropyl, cyclopropylmethyl, cyclopentyl, cyclopentylmethyl, cyclohexyl, cycloheptyl and norbornyl. “C₀-Cₙalkyl” refers to a single covalent bond (C₀) or an alkyl group having from 1 to n carbon atoms; for example, “C₀-C₆alkyl” refers to a single covalent bond or a C₁-C₆alkyl group.

Similarly, “alkenyl” refers to straight or branched chain alkene groups or cycloalkene groups, in which at least one unsaturated carbon-carbon double bond is present. Alkenyl groups include C₂-C₈alkenyl, C₂-C₆alkenyl and C₂-C₄alkenyl groups, which have from 2 to 8, 2 to 6 or 2 to 4 carbon atoms, respectively, such as ethenyl, allyl or isopropenyl. “Alkynyl” refers to straight or branched chain alkyne groups, which have one or more unsaturated carbon-carbon bonds, at least one of which is a triple bond. Alkynyl groups include C₂-C₈alkynyl, C₂-C₆alkynyl and C₂-C₄alkynyl groups, which have from 2 to 8, 2 to 6 or 2 to 4 carbon atoms, respectively. In certain embodiments, preferred alkenyl and alkynyl groups are straight or branched chain.

By “alkoxy,” as used herein, is meant an alkyl group as described above attached via an oxygen bridge. Alkoxy groups include C₁-C₈alkoxy, C₁-C₆alkoxy and C₁-C₄alkoxy groups, which have from 1 to 8, 1 to 6 or 1 to 4 carbon atoms, respectively. Alkoxy groups include, for example, methoxy, ethoxy, propoxy, isopropoxy, n-butoxy, sec-butoxy, tert-butoxy, n-pentoxy, 2-pentoxy, 3-pentoxy, isopentoxy, neopentoxy, hexoxy, 2-hexoxy, 3-hexoxy and 3-methylpentoxy. Similarly, “alkylthio” refers to an alkyl group as described above attached via a sulfur bridge.

The term “alkanoyl” refers to an acyl group in a linear or branched arrangement (e.g., —(C═O)-alkyl), where attachment is through the carbon of the keto group. Alkanoyl groups include C₂-C₈alkanoyl, C₂-C₆alkanoyl and C₂-C₄alkanoyl groups, which have from 2 to 8, 2 to 6 or 2 to 4 carbon atoms, respectively. “C₁alkanoyl” refers to —(C═O)—H, which (along with C₂-C₈alkanoyl) is encompassed by the term “C₁-C₈alkanoyl.”

An “alkanone” is a ketone group in which the carbon atoms are in a linear or branched alkyl arrangement. “C₃-C₈alkanone,” “C₃-C₆alkanone” and “C₃-C₄alkanone” refer to an alkanone having from 3 to 8, 6 or 4 carbon atoms, respectively. By way of example, a C₃ alkanone group has the structure —CH₂—(C═O)—CH₃.

Similarly, “alkyl ether” refers to a linear or branched ether substituent linked via a carbon-carbon bond. Alkyl ether groups include C₂-C₈alkyl ether, C₂-C₆alkyl ether and C₂-C₄alkyl ether groups, which have 2 to 8, 6 or 4 carbon atoms, respectively. By way of example, a C₂ alkyl ether group has the structure —CH₂—O—CH₃. A representative branched alkyl ether substituent is —C(CH₃)₂CH₂—O—CH₃.

The term “alkoxycarbonyl” refers to an alkoxy group linked via a carbonyl (i.e., a group having the general structure —C(═O)—O-alkyl). Alkoxycarbonyl groups include C₂-C₈, C₂-C₆ and C₂-C₄alkoxycarbonyl groups, which have from 2 to 8, 6 or 4 carbon atoms, respectively. “C₁alkoxycarbonyl” refers to —C(═O)—OH, which is encompassed by the term “C₁-C₈alkoxycarbonyl.”

“Alkanoyloxy,” as used herein, refers to an alkanoyl group linked via an oxygen bridge (i.e., a group having the general structure —O—C(═O)-alkyl). Alkanoyloxy groups include C₂-C₈, C₂-C₆ and C₂-C₄alkanoyloxy groups, which have from 2 to 8, 6 or 4 carbon atoms, respectively.

“Alkylamino” refers to a secondary or tertiary amine having the general structure —NH(alkyl) or —N(alkyl)(alkyl), wherein each alkyl may be the same or different. Such groups include, for example, mono- and di-(C₁-C₈alkyl)amino groups, in which each alkyl may be the same or different and may contain from 1 to 8 carbon atoms, as well as mono- and di-(C₁-C₆alkyl)amino groups and mono- and di-(C₁-C₄alkyl)amino groups.

“Alkylaminoalkyl” refers to an alkylamino group linked via an alkyl group (i.e., a group having the general structure -alkyl-NH-alkyl or -alkyl-N(alkyl)(alkyl)) in which each alkyl is selected independently. Such groups include, for example, mono- and di-(C₁-C₆alkyl)aminoC₁-C₆alkyl and mono- and di-(C₁-C₄alkyl)aminoC₁-C₄alkyl, in which each alkyl may be the same or different. “Mono- or di-(C₁-C₆alkyl)aminoC₀-C₆alkyl” refers to a mono- or di-(C₁-C₆alkyl)amino group linked via a direct bond or a C₁-C₆alkyl group. The following are representative alkylaminoalkyl groups:

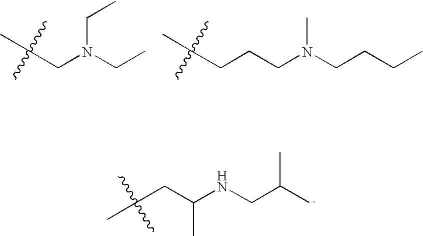

The term “aminocarbonyl” refers to an amide group (i.e., —(C═O)NH₂). “Mono- or di-(C₁-C₈alkyl)aminocarbonyl” is an aminocarbonyl group in which one or both of the hydrogen atoms is replaced with C₁-C₈alkyl. If both hydrogen atoms are so replaced, the C₁-C₈alkyl groups may be the same or different.

The term “halogen” refers to fluorine, chlorine, bromine and iodine.

A “haloalkyl” is a branched or straight-chain alkyl group, substituted with 1 or more halogen atoms (e.g., “C₁-C₈haloalkyl” groups have from 1 to 8 carbon atoms; “C₁-C₆haloalkyl” groups have from 1 to 6 carbon atoms). Examples of haloalkyl groups include, but are not limited to, mono-, di- or tri-fluoromethyl; mono-, di- or tri-chloromethyl; mono-, di-, tri-, tetra- or penta-fluoroethyl; mono-, di-, tri-, tetra- or penta-chloroethyl; and 1,2,2,2-tetrafluoro-1-trifluoromethyl-ethyl. Typical haloalkyl groups are trifluoromethyl and difluoromethyl.

“Haloalkoxy” indicates a haloalkyl group as defined above attached through an oxygen bridge. “C₁-C₈haloalkoxy” groups have from 1 to 8 carbon atoms.

A dash (“-”) that is not between two letters or symbols is used to indicate a point of attachment for a substituent. For example, —CONH₂ is attached through the carbon atom.

A “heteroatom,” as used herein, is oxygen, sulfur or nitrogen.

A “heterocycle” has from 1 to 3 fused, pendant or spiro rings, at least one of which is a heterocyclic ring (i.e., one or more ring atoms is a heteroatom, with the remaining ring atoms being carbon). Typically, a heterocyclic ring comprises 1, 2, 3 or 4 heteroatoms; within certain embodiments each heterocyclic ring has 1 or 2 heteroatoms per ring. Each heterocyclic ring generally contains from 3 to 8 ring members (rings having from 4 or 5 to 7 ring members are recited in certain embodiments) and heterocycles comprising fused, pendant or spiro rings typically contain from 9 to 14 ring members. Certain heterocycles comprise a sulfur atom as a ring member; in certain embodiments, the sulfur atom is oxidized to SO or SO₂. Heterocycles may be optionally substituted with a variety of substituents, as indicated. Unless otherwise specified, a heterocycle may be a heterocycloalkyl group (i.e., each ring is saturated or partially saturated) or a heteroaryl group (i.e., at least one ring within the group is aromatic). A heterocyclic group may generally be linked via any ring or substituent atom, provided that a stable compound results. N-linked heterocyclic groups are linked via a component nitrogen atom.

A “heterocycleC₀-C₄alkyl” is a heterocyclic group linked via a direct bond or C₁-C₄alkyl group. A (4- to 7-membered heterocycle)C₀-C₄alkyl is a heterocyclic group having from 4 to 7 ring members linked via a direct bond or an alkyl group having from 1 to 4 carbon atoms.

Certain heterocyclic groups are 4- to 7-membered or 5- to 7-membered groups that are optionally substituted. 4- to 7-membered heterocycloalkyl groups include, for example, piperidinyl, piperazinyl, pyrrolidinyl, azepanyl, morpholino, thiomorpholino and 1,1-dioxo-thiomorpholin-4-yl. Such groups may be substituted as indicated. Representative aromatic heterocycles are azocinyl, pyridyl, pyrimidyl, imidazolyl and tetrazolyl.

“Cycloalkyl” refers to saturated and/or partially saturated rings in which all ring members are carbon, such as cyclopropyl, cyclobutyl, cyclopentyl, cyclohexyl, cycloheptyl, cyclooctyl, adamantyl, decahydro-naphthalenyl, octahydro-indenyl, and partially saturated variants of any of the foregoing, such as cyclohexenyl. Certain cycloalkyl groups are C₃-C₇cycloalkyl, in which the ring contains from 3 to 7 ring members, all of which are carbon. A “C₃-C₇cycloalkyl(C₀-C₄alkyl)” is a C₃-C₇cycloalkyl group linked via a single covalent bond or a C₁-C₄alkylene group.

“PhenylC₀-C₄alkyl” is a phenyl group that is either directly attached to a molecular moiety via a single covalent bond to through a 1 to 4 carbon alkyl linker. Similarly “phenylC₀-C₄alkoxy” is a phenyl group that that is either directly attached to a molecular moiety via a single covalent bond to through a 1 to 4 carbon alkoxy linker, where the point of attachment is on the oxygen atom of the alkoxy.

A “substituent,” as used herein, refers to a molecular moiety that is covalently bonded to an atom within a molecule of interest. For example, a ring substituent may be a moiety such as a halogen, alkyl group, haloalkyl group or other group discussed herein that is covalently bonded to an atom (preferably a carbon or nitrogen atom) that is a ring member. Substituents or aromatic groups are generally covalently bonded to a ring carbon atom. The term “substitution” refers to replacing a hydrogen atom in a molecular structure with a substituent, such that the valence on the designated atom is not exceeded, and such that a chemically stable compound (i.e., a compound that can be isolated, characterized and tested for biological activity) results from the substitution.

Groups that are “optionally substituted” are unsubstituted or are substituted by other than hydrogen at one or more available positions, typically 1, 2, 3, 4 or 5 positions, by one or more suitable groups (which may be the same or different). Such optional substituents include, for example, hydroxy, halogen, cyano, nitro, C₁-C₈alkyl, C₂-C₈alkenyl, C₂-C₈alkynyl, C₁-C₈alkoxy, C₂-C₈alkyl ether, C₃-C₈alkanone, C₁-C₈alkylthio, amino, mono- or di-(C₁-C₈alkyl)amino, C₁-C₈haloalkyl, C₁-C₈haloalkoxy, C₁-C₈alkanoyl, C₂-C₈alkanoyloxy, C₁-C₈alkoxycarbonyl, —COOH, —CONH₂, mono- or di-(C₁-C₈alkyl)aminocarbonyl, —SO₂NH₂, and/or mono or di(C₁-C₈alkyl)sulfonamido, as well as carbocyclic and heterocyclic groups. Optional substitution is also indicated by the phrase “substituted with from 0 to X substituents,” where X is the maximum number of possible substituents. Certain optionally substituted groups are substituted with from 0 to 2, 3 or 4 independently selected substituents (i.e., are unsubstituted or substituted with up to the recited maximum number of substitutents).

The term “MCH receptor” refers to any naturally-occurring mammalian (especially human, monkey or canine) MCH type 1 or type 2 receptor, as well as chimeric receptors in which one or more domains of a naturally-occurring MCH1R or MCH2R are replaced with a corresponding domain of a different G protein-coupled receptor, such that the ability of the chimeric receptor to bind MCH and mediate a dose-dependent release of intracellular calcium is not diminished. MCH receptors for use within the various assays and other methods described herein include, for example, recombinantly expressed human MCH receptor (e.g., Genbank Accession No. Z86090; SEQ ID NO:29 of U.S. Patent Application Publication Number 2003/0148457), monkey MCH receptor (e.g., SEQ ID NO:2, 34 or 36 of U.S. Patent Application Publication Number 2003/0114644) or canine MCH receptor (e.g., SEQ ID NO:39 of U.S. Patent Application Publication Number 2003/0114644). Chimeric MCH receptors that may be used as described herein include, for example, those disclosed in U.S. Patent Application Publication Numbers 2003/0114644 and 2003/0148457.

A “MCH receptor modulator,” also referred to herein as a “modulator,” is a compound that alters (increases or decreases) MCH receptor activation and/or MCH receptor-mediated signal transduction. MCH receptor modulators specifically provided herein are compounds of Formula I and pharmaceutically acceptable salts of such compounds. A modulator may be a MCH receptor agonist or antagonist. In certain embodiments, a MCH receptor modulator may exhibit an EC₅₀ or IC₅₀ at MCH receptor that is less than 1 micromolar, 500 nM, 200 nM, 100 nM, 50 nM, 25 nM or 10 nM in a standard calcium mobilization assay (as described in Example 3, herein). A modulator may be a MCH receptor agonist or antagonist, although, for certain purposes described herein, a modulator preferably inhibits MCH receptor activation resulting from binding of MCH (i.e., the modulator is an antagonist).

A MCH receptor modulator binds with “high affinity” if the Kᵢ at a MCH receptor is less than 1 micromolar, preferably less than 500 nanomolar, 100 nanomolar or 10 nanomolar. A modulator binds “specifically” to MCH receptor if it binds to a MCH receptor (total binding minus nonspecific binding) with a Kᵢ that is 10-fold, preferably 100-fold, and more preferably 1000-fold, less than the Kᵢ measured for modulator binding to other G protein-coupled receptors. For example, a modulator may have a Kᵢ of 500 nanomolar or less in an MCH receptor ligand binding assay and a Kᵢ of at least 1 micromolar in a dopamine receptor ligand binding assay, such as the assay described in Example 4 (pages 111-112) of PCT International Publication Number WO 02/094799, which is hereby incorporated by reference. A representative assay for determining Kᵢ at MCH receptor is provided in Example 2, herein.

A modulator is considered an “antagonist” if it detectably inhibits MCH binding to MCH receptor and/or MCH-mediated signal transduction (using, for example, the representative assay provided in Example 3); in general, such an antagonist has a IC₅₀ value of less than 1 micromolar, preferably less than 100 nanomolar, and more preferably less than 10 nanomolar or 1 nanomolar within the assay provided in Example 3. MCH receptor antagonists include neutral antagonists and inverse agonists.

An “inverse agonist” is a compound that reduces the activity of MCH receptor below its basal activity level in the absence of added ligand. Inverse agonists may also inhibit the activity of MCH at MCH receptor, and/or may also inhibit binding of MCH to MCH receptor. The ability of a compound to inhibit the binding of MCH to MCH receptor may be measured by a binding assay, such as the binding assay given in Example 2. The basal activity of MCH receptor, as well as the reduction in MCH receptor activity due to the presence of antagonist, may be determined from a calcium mobilization assay, such as the assay of Example 3.

A “neutral antagonist” of MCH receptor is a compound that inhibits the activity of MCH at MCH receptor, but does not significantly change the basal activity of the receptor (i.e., within a calcium mobilization assay as described in Example 3 performed in the absence of ligand, MCH receptor activity is reduced by no more than 10%, more preferably by no more than 5%, and even more preferably by no more than 2%; most preferably, there is no detectable reduction in activity). Neutral antagonists may also inhibit ligand binding of ligand to MCH receptor.

As used herein a “MCH receptor agonist” is a compound that elevates the activity of the receptor above the basal activity level of the receptor (i.e., enhances MCH receptor activation and/or MCH receptor-mediated signal transduction). MCH receptor agonist activity may be identified using the representative assay provided in Example 3. In general, such an agonist has an EC₅₀ value of less than 1 micromolar, preferably less than 100 nanomolar, and more preferably less than 10 nanomolar within the assay provided in Example 3.

A “therapeutically effective amount” (or dose) is an amount that, upon administration, is sufficient to provide a discernible patient benefit. For example, a therapeutically effective amount may reduce symptom severity or frequency, or to result in detectable weight loss. Alternatively, or in addition, a therapeutically effective amount may improve patient status or outcome and/or prevent or delay disease or symptom onset. A therapeutically effective amount or dose generally results in a concentration of compound in a body fluid (such as blood, plasma, serum, CSF, synovial fluid, lymph, cellular interstitial fluid, tears or urine) that is sufficient to alter the binding of ligand to MCH receptor in vitro (using the assay provided in Example 8) and/or MCH-mediated signal transduction (using an assay provided in Example 9).

A “disease or disorder associated with MCH receptor activation,” as used herein is any condition that is characterized by inappropriate stimulation of MCH receptor, regardless of the amount of MCH present locally, and/or that is responsive to modulation of MCH receptor activity (i.e., the condition or a symptom thereof is alleviated by such modulation). Such conditions include, for example, metabolic disorders (such as diabetes), heart disease, stroke, eating disorders (such as obesity and bulimia nervosa) and sexual disorders such as anorgasmic and psychogenic impotence, as well as other diseases and disorders recited herein.

A “patient” is any individual treated with a MCH modulator as provided herein. Patients include humans, as well as other animals such as companion animals (e.g., dogs and cats) and livestock. Patients may be experiencing one or more symptoms of a condition responsive to MCH receptor modulation, or may be free of such symptom(s) (i.e., treatment may be prophylactic).

### Substituted Tetrahydroisoquinoline Analogues

As noted above, the present invention provides compounds of Formula I, and pharmaceutically acceptable salts thereof, in which variables are generally as described above.

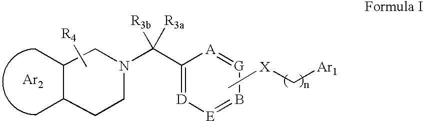

In certain embodiments, each R₂ is independently chosen from hydrogen, halogen, amino, hydroxy, cyano, nitro, C₁-C₄alkyl, C₁-C₄alkoxy, C₁-C₄haloalkyl and C₁-C₄haloalkoxy.

The present invention also includes compounds and salts of Formula II and Formula III:

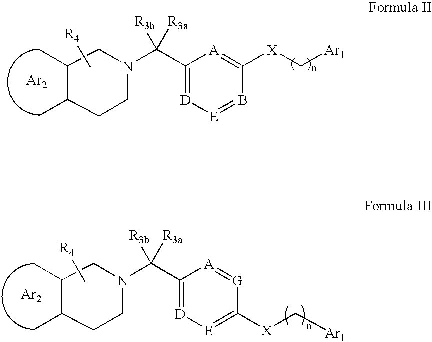

Within Formula II and Formula III, variables are as described above. In certain embodiments A, E, D, B and G are each CR₂. In other embodiments, 1 or 2 of A, E, D and B (Formula II) or G (Formula III) is N, and the remainder are CR₂ (e.g., in certain compounds of Formula II, A or B is N).

In some embodiments described herein, n is 0; in other embodiments, n is 1.

### The Ar₁ Variable

Within certain compounds and pharmaceutically acceptable salts of Formula I-III, Ar₁ is phenyl or pyridyl, each of which is substituted with from 0 to 4 substituents independently chosen from hydroxy, halogen, cyano, nitro, amino, C₁-C₆alkyl, C₃-C₇cycloalkyl(C₀-C₄alkyl), C₁-C₆haloalkyl, C₁-C₆alkoxy, C₁-C₆haloalkoxy, C₁-C₆alkylthio, C₂-C₆alkyl ether, mono- and di-(C₁-C₆alkyl)amino, phenyl and phenoxy.

In certain embodiments, Ar₁ is substituted with 1, 2 or 3 substituents independently chosen from hydroxy, halogen, cyano, nitro, amino, C₁-C₄alkyl, C₁-C₄alkoxy, C₃-C₇cycloalkyl(C₀-C₂alkyl), C₁-C₄haloalkyl, C₁-C₄haloalkoxy and phenoxy.

### The Ar₂ Variable

Within certain compounds

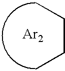

represents a fused ring chosen from phenyl and pyridyl, each of which is substituted with from 0 to 4 substituents independently chosen from hydroxy, halogen, cyano, nitro, amino, C₁-C₆alkyl, C₁-C₆alkoxy, C₃-C₇cycloalkyl(C₀-C₄alkyl), C₁-C₆haloalkyl, C₁-C₆haloalkoxy, C₁-C₆alkylthio, C₂-C₆alkyl ether and mono- and di-(C₁-C₆alkyl)amino.

In certain embodiments,

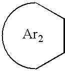

is substituted with 1, 2 or 3 substituents (e.g., independently chosen from hydroxy, halogen, cyano, nitro, amino, C₁-C₄alkyl, C₁-C₄alkoxy, C₃-C₇cycloalkyl(C₀-C₂alkyl) and C₁-C₄alkylthio).

### The R₂ Variable

Within certain compounds of Formula I-III, each R₂ is independently chosen from hydrogen, halogen, amino, hydroxy, cyano, nitro, C₁-C₄alkyl, C₁-C₄alkoxy, C₃-C₇cycloalkyl(C₀-C₂alkyl), C₁-C₄haloalkyl and C₁-C₄haloalkoxy. In certain exemplary embodiments, R₂ is independently chosen at each occurrence from hydrogen and methyl.

### The R₃ Variable

Within certain compounds of Formula I-III, R₃ₐ is hydrogen, C₁-C₄alkyl, C₂-C₄alkenyl, C₂-C₄alkynyl, C₃-C₇cycloalkyl(C₀-C₂alkyl) or C₁-C₄haloalkyl; and R_{3b} is hydrogen. In other embodiments, R₃ₐ is hydrogen or methyl; and R_{3b} is hydrogen. In further embodiments, R₃ₐ and R_{3b} are taken together to form an oxo group.

### The R₄ Variable

Within certain compounds of Formula I-III, R₄ represents 0, 1 or 2 substituents independently chosen from hydroxy, halogen, cyano, C₁-C₄alkyl, C₂-C₄alkenyl, C₂-C₄alkynyl, C₁-C₄haloalkyl, C₁-C₄alkoxy, C₃-C₇cycloalkyl(C₀-C₂alkyl) and C₁-C₄haloalkoxy. In further such compounds, R₄ represents 0, 1 or 2 substituents independently chosen from hydroxy, halogen, cyano, C₁-C₄alkyl, C₂-C₄alkenyl, C₂-C₄alkynyl, C₁-C₄alkoxy, C₃-C₇cycloalkyl(C₀-C₂alkyl), C₁-C₄haloalkyl and C₁-C₄haloalkoxy. In certain exemplary embodiments, R₄ represents 0 substituents or 1 substituent chosen from methyl, ethyl and methoxy.

In further embodiments, certain compounds of Formula I also satisfy Formula IV:

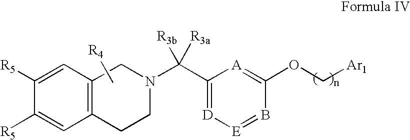

or are a pharmaceutically acceptable salt thereof. Within Formula IV:
Ar₁, n, R₄, A, B, D and E are as defined for Formula I; preferably each R₂ is independently chosen from hydrogen, halogen, amino, hydroxy, cyano, nitro, C₁-C₄alkyl, C₁-C₄alkoxy, C₃-C₇cycloalkyl(C₀-C₂alkyl), C₁-C₄haloalkyl and C₁-C₄haloalkoxy;
R₃ₐ and R_{3b} are as described for Formula I; preferably R₃ₐ is hydrogen or methyl, and R_{3b} is hydrogen; and
each R₅ is independently chosen from hydrogen, hydroxy, halogen, cyano, nitro, amino, C₁-C₆alkyl, C₁-C₆haloalkyl, C₁-C₆alkoxy, C₃-C₇cycloalkyl(C₀-C₂alkyl), C₁-C₆haloalkoxy, C₁-C₆alkylthio, C₂-C₆alkyl ether and mono- and di-(C₁-C₆alkyl)amino.
   Certain compounds of Formula I or II further satisfy Formula V:

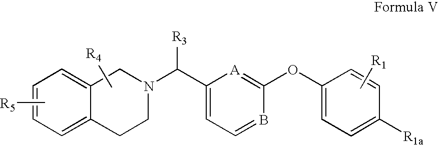

or are a pharmaceutically acceptable salt thereof. Within Formula V:
A and B are as defined for Formula I; preferably each R₂ is independently chosen from hydrogen, halogen, amino, hydroxy, cyano, nitro, C₁-C₄alkyl, C₁-C₄alkoxy, C₃-C₇cycloalkyl(C₀-C₂alkyl), C₁-C₄haloalkyl and C₁-C₄haloalkoxy. Within certain embodiments, one of A and B is nitrogen; in other embodiments A and B are each CH.
R₁ₐ is hydrogen, hydroxy, halogen, cyano, nitro, amino, C₁-C₆alkyl, C₁-C₆haloalkyl, C₁-C₆alkoxy, C₃-C₇cycloalkyl(C₀-C₂alkyl), C₁-C₆haloalkoxy, C₁-C₆alkylthio, C₂-C₆alkyl ether, mono- or di-(C₁-C₆alkyl)amino, phenyl or phenoxy. In certain embodiments, R₁ₐ is not hydrogen. For example, R₁ₐ may be chosen from cyano, chloro, fluoro, C₁-C₄alkyl (e.g., methyl, ethyl, propyl, isopropyl, n-butyl or tert-butyl), C₁-C₄alkoxy (e.g., methoxy or ethoxy), C₃-C₇cycloalkyl(C₀-C₂alkyl), C₁-C₄haloalkyl (e.g., trifluoromethyl), C₁-C₄haloalkoxy and phenoxy. In other embodiments R₁ₐ is hydroxy, halogen, cyano, C₁-C₆alkyl, C₁-C₆alkoxy, C₁-C₆haloalkyl, C₁-C₆haloalkoxy or phenoxy.
R₁, represents from 0 to 3 substituents independently chosen from hydroxy, halogen, cyano, nitro, amino, C₁-C₆alkyl, C₁-C₆haloalkyl, C₁-C₆alkoxy, C₁-C₆haloalkoxy, C₁-C₆alkylthio, C₂-C₆alkyl ether, mono- and di-(C₁-C₆alkyl)amino, phenyl and phenoxy.
R₃ is hydrogen, C₁-C₆alkyl, C₂-C₆alkenyl, C₂-C₆alkynyl or C₁-C₆haloalkyl. In certain embodiments, R₃ is hydrogen, C₁-C₄alkyl, C₂-C₄alkenyl, C₂-C₄alkynyl or C₁-C₄haloalkyl; preferably R₃ is hydrogen, methyl or ethyl.
R₄ is as described for Formula I; preferably R₄ of Formula V represents 0, 1 or 2 substituents independently chosen from hydroxy, halogen, cyano, C₁-C₄alkyl (e.g., methyl or ethyl), C₁-C₄alkoxy, C₃-C₇cycloalkyl(C₀-C₂alkyl), C₂-C₄alkenyl, C₂-C₄alkynyl, C₁-C₄haloalkyl and C₁-C₄haloalkoxy.
R₅ represents from 0 to 4 substituents independently chosen from Rₐ, and preferably independently chosen from hydroxy, halogen, cyano, nitro, amino, C₁-C₆alkyl, C₁-C₆haloalkyl, C₁-C₆alkoxy, C₃-C₇cycloalkyl(C₀-C₂alkyl), C₁-C₆haloalkoxy, C₁-C₆alkylthio, C₂-C₆alkyl ether and mono- and di-(C₁-C₆alkyl)amino; or two adjacent R₅ are taken together to form, with the carbon atoms to which they are bound, a fused 5- or 6-membered ring substituted with from 0 to 4 substituents independently chosen from Rₐ. In certain embodiments, R₅ represents from 1 to 3 independently chosen substituents; or two adjacent R₅ are taken together to form, with the carbon atoms to which they are bound, a fused 5- or 6-membered heterocyclic ring (e.g., a 5-membered heterocycle such as dioxole).

In further embodiments, certain compounds further satisfy Formula VI, VII or VIII, or are a pharmaceutically acceptable salt of such a compound, wherein R₃ of Formula VII is hydrogen, C₁-C₆alkyl, C₂-C₆alkenyl, C₂-C₆alkynyl or C₁-C₆haloalkyl, and the other variables are as described for Formula V.

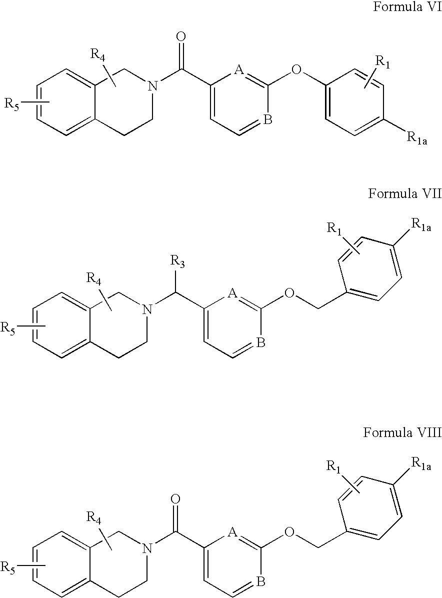

Certain compounds of Formula IV further satisfy Formula IX X, XI or XII, wherein the variables are generally as described for Formula V, and each R₅ is as described for Formula IV.

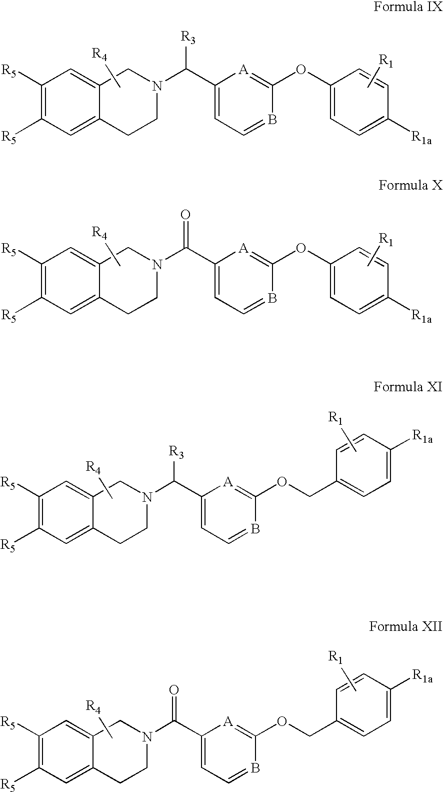

Other compounds of Formula I further satisfy Formula XIII, or are a pharmaceutically acceptable salt thereof, wherein R₃ₐ and R_{3b} are as described for Formula I and the remainder of the variables are as described for Formula V:

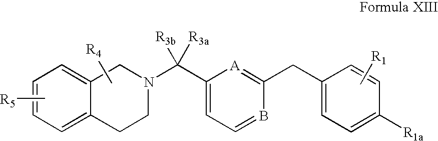

Certain compounds of Formula I further satisfy Formula XIV, or are a pharmaceutically acceptable salt thereof:

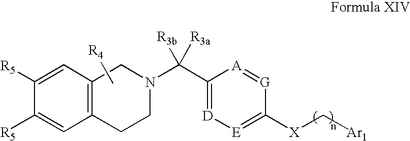

Within Formula XIV:
Ar₁, R₄, n, A, G, D and E are as defined for Formula I; in certain embodiments, at least one of A, G, D and E is nitrogen; preferably each R₂ is independently chosen from hydrogen, halogen, amino, hydroxy, cyano, nitro, C₁-C₄alkyl, C₁-C₄alkoxy, C₁-C₄haloalkyl and C₁-C₄haloalkoxy.
X is oxygen or CH₂.
R₃ₐ is hydrogen, hydroxy, halogen, cyano, C₁-C₆alkyl, C₂-C₆alkenyl, C₂-C₆alkynyl, C₁-C₆alkoxy, C₃-C₇cycloalkyl(C₀-C₄alkyl), C₁-C₆haloalkyl or C₁-C₆haloalkoxy.
R_{3b} is hydrogen, hydroxy, halogen, cyano, C₁-C₆alkyl, C₂-C₆alkenyl, C₂-C₆alkynyl, C₁-C₆alkoxy, C₃-C₇cycloalkyl(C₀-C₄alkyl), C₁-C₆haloalkyl or C₁-C₆haloalkoxy; or R₃ₐ and R_{3b} are taken together to form an oxo group; in certain embodiments, R₃ₐ and R_{3b} are not both hydrogen; preferably R₃ₐ is methyl and R_{3b} is hydrogen.
Each R₅ is independently chosen from hydrogen, hydroxy, halogen, cyano, nitro, amino, C₁-C₆alkyl, C₁-C₆haloalkyl, C₁-C₆alkoxy, C₃-C₇cycloalkyl(C₀-C₂alkyl), C₁-C₆haloalkoxy, C₁-C₆alkylthio, C₂-C₆alkyl ether and mono- and di-(C₁-C₆alkyl)amino.
   Certain compounds of Formula I further satisfy Formula XV:

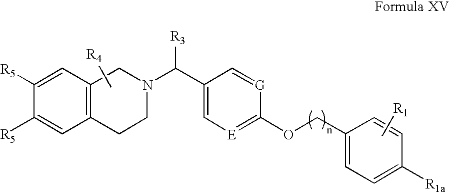

or are a pharmaceutically acceptable salt thereof. Within Formula XV:
G and E are as defined for Formula I; preferably each R₂ is independently chosen from hydrogen, halogen, amino, hydroxy, cyano, nitro, C₁-C₄alkyl, C₁-C₄alkoxy, C₃-C₇cycloalkyl(C₀-C₂alkyl), C₁-C₄haloalkyl and C₁-C₄haloalkoxy. In certain embodiments, at least one of G and E is nitrogen (e.g., one of G and E is nitrogen); in other embodiments, G and E are CH.
R₁ₐ is hydrogen, hydroxy, halogen, cyano, nitro, amino, C₁-C₆alkyl, C₁-C₆haloalkyl, C₁-C₆alkoxy, C₃-C₇cycloalkyl(C₀-C₄alkyl), C₁-C₆haloalkoxy, C₁-C₆alkylthio, C₂-C₆alkyl ether, mono- or di-(C₁-C₆alkyl)amino, phenyl or phenoxy; in certain embodiments R₁ₐ is hydroxy, halogen, cyano, C₁-C₆alkyl, C₁-C₆alkoxy, C₁-C₆haloalkyl, C₁-C₆haloalkoxy, or phenoxy. In further embodiments, R₁ₐ is not hydrogen. For example, R₁ₐ may be chosen from cyano, chloro, fluoro, C₁-C₄alkyl (e.g., methyl, ethyl, propyl, isopropyl, n-butyl or tert-butyl), C₁-C₄alkoxy (e.g., methoxy or ethoxy), C₃-C₇cycloalkyl(C₀-C₂alkyl), C₁-C₄haloalkyl (e.g., trifluoromethyl), C₁-C₄haloalkoxy and phenoxy.
R₁ represents from 0 to 3 substituents independently chosen from hydroxy, halogen, cyano, nitro, amino, C₁-C₆alkyl, C₁-C₆haloalkyl, C₁-C₆alkoxy, C₃-C₇cycloalkyl(C₀-C₄alkyl), C₁-C₆haloalkoxy, C₁-C₆alkylthio, C₂-C₆alkyl ether, mono- and di-(C₁-C₆alkyl)amino, phenyl and phenoxy.
R₃ is hydrogen, C₁-C₆alkyl, C₂-C₆alkenyl, C₂-C₆alkynyl or C₁-C₆haloalkyl. In certain embodiments, R₃ is hydrogen, C₁-C₄alkyl, C₂-C₄alkenyl, C₂-C₄alkynyl or C₁-C₄haloalkyl; preferably R₃ is hydrogen, methyl or ethyl.
R₄ is as described for Formula I; preferably R₄ of Formula V represents 0, 1 or 2 substituents independently chosen from hydroxy, halogen, cyano, C₁-C₄alkyl (e.g., methyl or ethyl), C₂-C₄alkenyl, C₂-C₄alkynyl, C₁-C₄alkoxy, C₃-C₇cycloalkyl(C₀-C₂alkyl), C₁-C₄haloalkyl and C₁-C₄haloalkoxy.
Each R₅ is independently chosen from hydrogen, hydroxy, halogen, cyano, nitro, amino, C₁-C₆alkyl, C₁-C₆haloalkyl, C₁-C₆alkoxy, C₃-C₇cycloalkyl(C₀-C₂alkyl), C₁-C₆haloalkoxy, C₁-C₆alkylthio, C₂-C₆alkyl ether and mono- and di-(C₁-C₆alkyl)amino; or both R₅ are taken together to form, with the carbon atoms to which they are bound, a fused 5- or 6-membered ring substituted with from 0 to 4 substituents independently chosen from Rₐ.

Representative compounds of Formulas I-XV include, but are not limited to, those specifically described in Example 1. It will be apparent that the compounds recited therein are representative only, and are not intended to limit the scope of the present invention. Further, as noted above, all compounds of the present invention may be present as free base or as a pharmaceutically acceptable salt.

In certain embodiments, compounds provided herein detectably alter (modulate) MCH binding to MCHR1 and/or MCHR2 receptor, as determined using a standard in vitro MCH receptor binding assay and/or calcium mobilization assay. References herein and in the claims to a “MCH receptor ligand binding assay” refer to the standard in vitro receptor binding assay provided in Example 2. Briefly, a competition assay is performed in which an MCH receptor preparation is incubated with labeled (e.g., ¹²⁵I or ³H) MCH and unlabeled test compound. Within the assays provided herein, the MCH receptor used is preferably a mammalian MCHR1 or MCHR2 receptor, more preferably a human, monkey or canine MCHR1 or MCHR2 receptor, most preferably human MCHR1. Incubation with a compound that detectably modulates MCH binding to MCH receptor will result in a decrease or increase in the amount of label bound to the MCH receptor preparation, relative to the amount of label bound in the absence of the compound. Preferably, such a compound will exhibit a Kᵢ at an MCH receptor of less than 1 micromolar, more preferably less than 500 nM, 100 nM, 20 nM or 10 nM, within a MCH receptor ligand binding assay performed as described in Example 2. Certain preferred compounds are MCH receptor antagonists, and exhibit IC₅₀ values of about 4 micromolar or less, more preferably 1 micromolar or less, still more preferably about 100 nanomolar or less, 10 nanomolar or less or 1 nanomolar or less within a standard in vitro MCH receptor mediated calcium mobilization assay, as provided in Example 3.

If desired, MCH receptor modulators provided herein may be evaluated for certain pharmacological properties including, but not limited to, oral bioavailability (preferred compounds are orally bioavailable to an extent allowing for therapeutically effective concentrations of the compound to be achieved at oral doses of less than 140 mg/kg, preferably less than 50 mg/kg, more preferably less than 30 mg/kg, even more preferably less than 10 mg/kg, still more preferably less than 1 mg/kg), toxicity (a preferred MCH receptor modulator is nontoxic when a therapeutically effective amount is administered to a subject), side effects (a preferred MCH receptor modulator produces side effects comparable to placebo when a therapeutically effective amount of the compound is administered to a subject), serum protein binding and in vitro and in vivo half-life (a preferred MCH receptor modulator exhibits an in vitro half-life that is equal to an in vivo half-life allowing for Q.I.D. dosing, preferably T.I.D. dosing, more preferably B.I.D. dosing, and most preferably once-a-day dosing). In addition, differential penetration of the blood brain barrier may be desirable for MCH receptor modulators used to treat CNS disorders, while low brain levels of MCH receptor modulators used to treat peripheral disorders are preferred. Routine assays that are well known in the art may be used to assess these properties and identify superior compounds for a particular use. For example, assays used to predict bioavailability include transport across human intestinal cell monolayers, including Caco-2 cell monolayers. Penetration of the blood brain barrier of a compound in humans may be predicted from the brain levels of the compound in laboratory animals given the compound (e.g., intravenously). Serum protein binding may be predicted from albumin binding assays. Compound half-life is inversely proportional to the frequency of dosage of a compound. In vitro half-lives of compounds may be predicted from assays of microsomal half-life as described within Example 11, herein.

As noted above, preferred MCH receptor modulators provided herein are nontoxic. In general, the term “nontoxic” as used herein shall be understood in a relative sense and is intended to refer to any substance that has been approved by the United States Food and Drug Administration (“FDA”) for administration to mammals (preferably humans) or, in keeping with established criteria, is susceptible to approval by the FDA for administration to mammals (preferably humans). In addition, a highly preferred nontoxic compound generally satisfies one or more of the following criteria when administered in minimum therapeutically effective amounts, or when contacted with cells at a concentration that is sufficient to inhibit the binding of MCH receptor ligand to MCH receptor in vitro: (1) does not substantially inhibit cellular ATP production; (2) does not significantly prolong heart QT intervals; (3) does not cause substantial liver enlargement and (4) does not cause substantial release of liver enzymes.

As used herein, a compound that does not substantially inhibit cellular ATP production is a compound that satisfies the criteria set forth in Example 10, herein. In other words, cells treated as described in Example 10 with 100 μM of such a compound exhibit ATP levels that are at least 50% of the ATP levels detected in untreated cells. In more highly preferred embodiments, such cells exhibit ATP levels that are at least 80% of the ATP levels detected in untreated cells.

A compound that does not significantly prolong heart QT intervals is a compound that does not result in a statistically significant prolongation of heart QT intervals (as determined by electrocardiography) in guinea pigs, minipigs or dogs upon administration of a dose that yields a serum concentration equal to the EC₅₀ or IC₅₀ for the compound. In certain preferred embodiments, a dose of 0.01, 0.05, 0.1, 0.5, 1, 5, 10, 40 or 50 mg/kg administered parenterally or orally does not result in a statistically significant prolongation of heart QT intervals. By “statistically significant” is meant results varying from control at the p<0.1 level or more preferably at the p<0.05 level of significance as measured using a standard parametric assay of statistical significance such as a student's T test.

A compound does not cause substantial liver enlargement if daily treatment of laboratory rodents (e.g., mice or rats) for 5-10 days with a dose that yields a serum concentration equal to the EC₅₀ or IC₅₀ for the compound results in an increase in liver to body weight ratio that is no more than 100% over matched controls. In more highly preferred embodiments, such doses do not cause liver enlargement of more than 75% or 50% over matched controls. If non-rodent mammals (e.g., dogs) are used, such doses should not result in an increase of liver to body weight ratio of more than 50%, preferably not more than 25%, and more preferably not more than 10% over matched untreated controls. Preferred doses within such assays include 0.01, 0.05, 0.1, 0.5, 1, 5, 10, 40 or 50 mg/kg administered parenterally or orally.

Similarly, a compound does not promote substantial release of liver enzymes if administration of twice the minimum dose that yields a serum concentration equal to the EC₅₀ or IC₅₀ for the compound does not elevate serum levels of ALT, LDH or AST in laboratory rodents by more than 100% over matched mock-treated controls. In more preferred embodiments, such doses do not elevate such serum levels by more than 75% or 50% over matched controls. Alternatively, a compound does not promote substantial release of liver enzymes if, in an in vitro hepatocyte assay, concentrations (in culture media or other such solutions that are contacted and incubated with hepatocytes in vitro) that are equal to the EC₅₀ or IC₅₀ for the compound do not cause detectable release of any of such liver enzymes into culture medium above baseline levels seen in media from matched mock-treated control cells. In more highly preferred embodiments, there is no detectable release of any of such liver enzymes into culture medium above baseline levels when such compound concentrations are five-fold, and preferably ten-fold the EC₅₀ or IC₅₀ for the compound.

In other embodiments, certain preferred compounds do not inhibit or induce microsomal cytochrome P450 enzyme activities, such as CYP1A2 activity, CYP2A6 activity, CYP2C9 activity, CYP2C19 activity, CYP2D6 activity, CYP2E1 activity or CYP3A4 activity at a concentration equal to the EC₅₀ or IC₅₀ for the compound.

Certain preferred compounds are not clastogenic (e.g., as determined using a mouse erythrocyte precursor cell micronucleus assay, an Ames micronucleus assay, a spiral micronucleus assay or the like) at a concentration equal the EC₅₀ or IC₅₀ for the compound. In other embodiments, certain preferred MCH receptor modulators do not induce sister chromatid exchange (e.g., in Chinese hamster ovary cells) at such concentrations.

For detection purposes, as discussed in more detail below, MCH receptor modulators provided herein may be isotopically-labeled or radiolabeled. For example, compounds of Formula I may have one or more atoms replaced by an atom of the same element having an atomic mass or mass number different from the atomic mass or mass number usually found in nature. Examples of isotopes that can be present in the compounds provided herein include isotopes of hydrogen, carbon, nitrogen, oxygen, phosphorous, fluorine and chlorine, such as ²H, 3H, ¹¹C, 13C, ¹⁴C, ¹⁵N, ¹⁸O, ¹⁷O, ³¹P, ³²P, ³⁵S, ¹⁸F and ³⁶Cl. In addition, substitution with heavy isotopes such as deuterium (i.e., ²H) can afford certain therapeutic advantages resulting from greater metabolic stability, for example increased in vivo half-life or reduced dosage requirements and, hence, may be preferred in some circumstances.

### Preparation of Compounds

Compounds provided herein may generally be prepared using standard synthetic methods. Starting materials are generally readily available from commercial sources, such as Sigma-Aldrich Corp. (St. Louis, Mo.). For example, a synthetic route similar to that shown in any one of Schemes A-E may be used. It will be apparent that the final product and any intermediate(s) shown in the following schemes may be extracted, dried, filtered and/or concentrated, and may be further purified (e.g., by chromatography). Each variable (e.g., “R”) in the following Schemes, refers to any group consistent with the description of the compounds provided herein. An individual skilled in the art may find modifications of one or several of the synthetic steps described herein without diverting significantly from the overall synthetic scheme. Further experimental details for synthesis of representative compounds via these schemes are provided in Example 1, herein.

In the following Schemes, the following abbreviations are used:

AcOH acetic acid

BOP benzotriazol-1-yl-oxy-tris(dimethylamino)phosphonium hexafluorophosphate

cat. catalytic

Cu(OAc)₂ copper (II) acetate

DCE 1,2-dichloroethane

DMF dimethylformamide

DMSO dimethylsulfoxide

EtOAc ethyl acetate

EtOH ethanol

MeOH methanol

NEt₃ triethylamine

THF tetrahydrofuran

TMSCl chlorotrimethylsilane

Tetrahydroisoquinoline analogue fragments for use in the following Schemes and Example 1 may be prepared using a variety of synthetic methods, such as

1. Pictet-Spengler (1983) J. Org. Chem. 48: 1932:

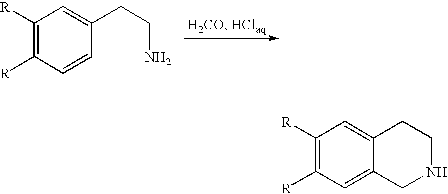

2. Electron-deficient ring system (Perchonock, et al. (1980) J. Org. Chem. 45: 1950-1953):

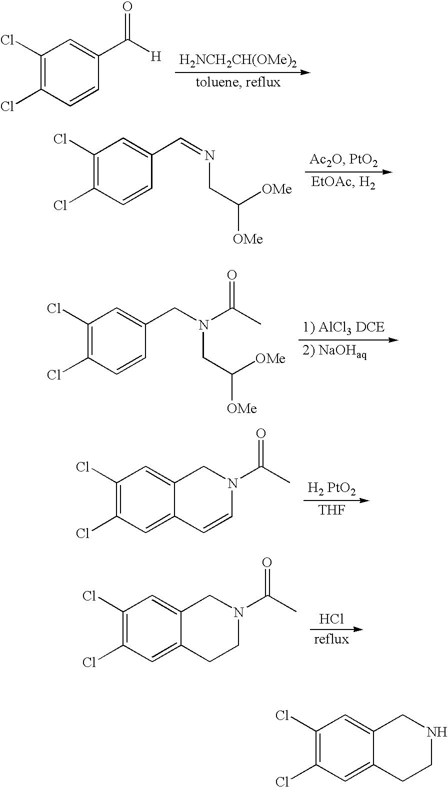

3. 1,1-Dimethyl THIQ Synthesis (Rodger et al. (1979) J. Med. Chem. 22: 117-119):

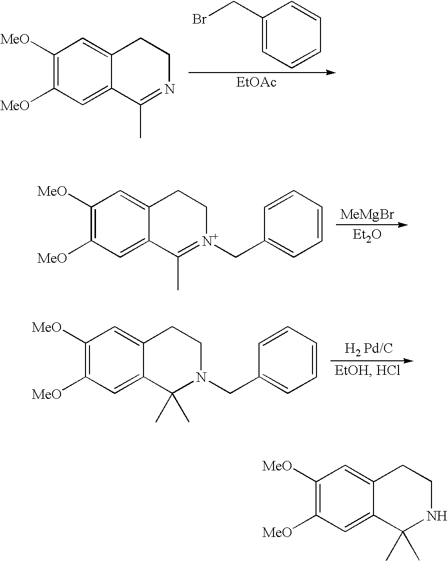

It will be apparent that the above methods may be modified in starting material and reagents so as to generate a variety of analogs with different

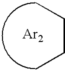

groups and substituents.

### General Synthetic Methods

The following Schemes which, illustrate representative synthetic methods for compounds of Formula I, wherein the variables are as defined above.

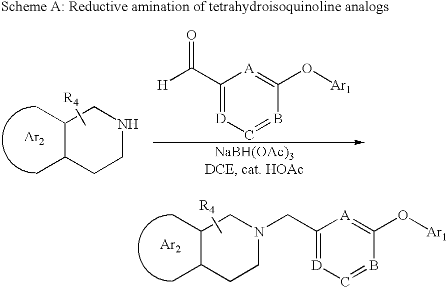

As shown in Scheme A, certain compounds of Formula I can be prepared by reductive amination of a tetrahydroisoquinoline derivative with an appropriate aldehyde. Such reactions are generally carried out in a solvent such as dichloromethane, 1,2-dichloroethane, tetrahydrofuran, toluene, benzene, methanol, ethanol or water, and are generally performed at a temperature between −78° C. and 150° C., preferably between 0° C. and 25° C., in the presence of a reducing agent (e.g., sodium borohydride, lithium borohydride, potassium borohydride, sodium cyanoborohydride or sodium triacetoxyborohydride) and in the presence of a catalyst such as hydrochloric acid, sulfuric acid or acetic acid.

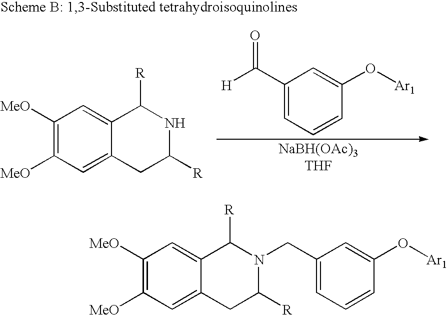

Scheme B illustrates the preparation of certain 1,3-substituted tetrahydroisoquinolines of Formula I by reductive amination of an appropriately substituted tetrahydroisoquinoline derivative with an appropriate aldehyde. Such reactions are generally carried out in a solvent such as dichloromethane, 1,2-dichloroethane, tetrahydrofuran, toluene, benzene, methanol, ethanol or water, and are generally performed at a temperature between −78° C. and 150° C., preferably between 0° C. and 25° C., in the presence of a reducing agent (e.g., sodium borohydride, lithium borohydride, potassium borohydride, sodium cyanoborohydride or sodium triacetoxyborohydride) and in the presence of a catalyst such as hydrochloric acid, sulfuric acid or acetic acid.

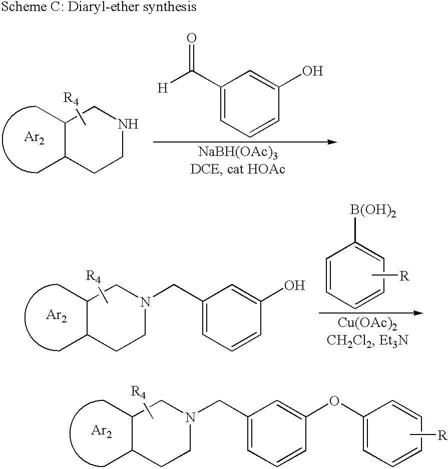

Scheme C illustrates the preparation of certain compounds of Formula I by reaction of an appropriately substituted boronic acid (or derivative thereof) with an appropriately substituted phenol. Such reactions are generally carried out in a solvent such as dichloromethane, 1,2-dichloroethane, tetrahydrofuran or toluene, and are generally performed at a temperature between −78° C. and 150° C., preferably between 0° C. and 25° C., in the presence of a catalyst (e.g., copper(II) acetate, or copper(II) chloride) and a base (e.g., triethylamine, diisopropylethylamine or N-methylpiperidine), and optionally in the presence of air. Compounds of Formula I may also be prepared using the Ullman ether synthesis (Whitesides et al. (1974) J. Am. Chem. Soc. 96:2829).

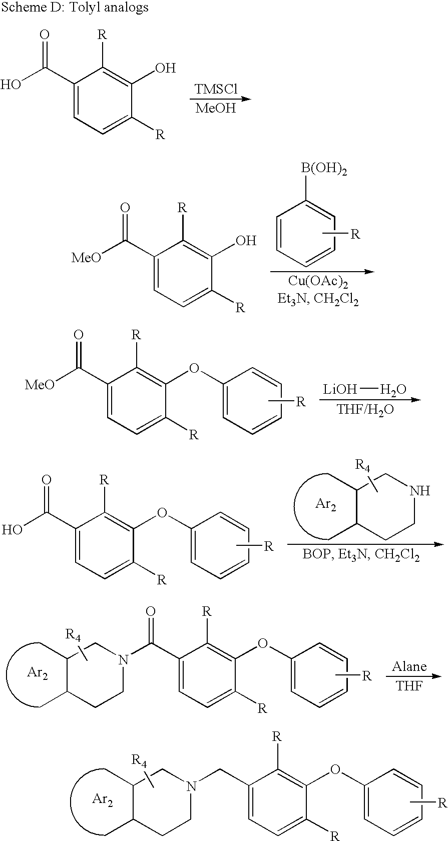

As shown in Scheme D, certain compounds of Formula I can be prepared by reduction of an appropriate amide with a reducing agent. Such reactions are generally carried out in a solvent such as ethyl ether, tetrahydrofuran, toluene, benzene or dimethoxyethane, and are generally performed at a temperature between 0° C. and 120° C., preferably between 25° C. and 80° C., using a reducing agent (e.g., lithium aluminum hydride, alane or borane).

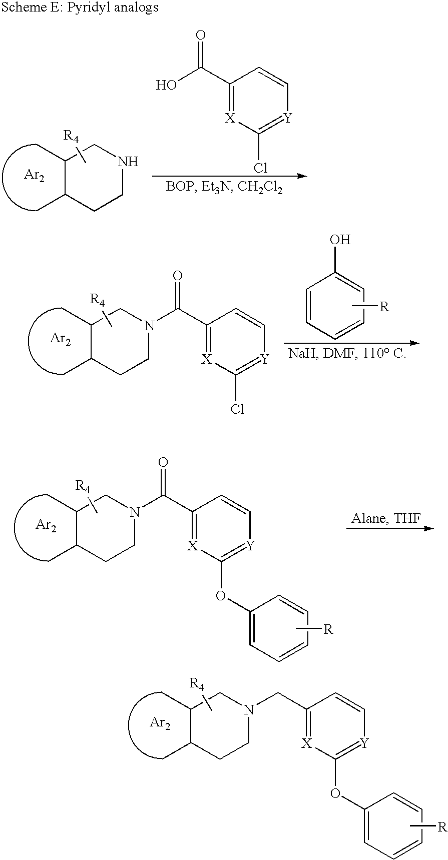

Similarly, as shown in Scheme E, certain heterocyclic compounds of Formula I can be prepared by reduction of an appropriate heterocyclic amide with a reducing agent. Such reactions are generally carried out in a solvent such as ethyl ether, tetrahydrofuran, toluene, benzene or dimethoxyethane, and are generally performed at a temperature between 0° C. and 120° C., preferably between 25° C. and 80° C., using a reducing agent (e.g., lithium aluminum hydride, alane or borane). The corresponding heterocyclic amides may be obtained by reacting a phenol with an appropriate haloheterocycle. Such reactions are generally carried out in a solvents such as N,N-dimethylformamide, N,N-dimethylacetamide, dimethylsulfoxide, sulfolane, acetonitrile, propionitrile or tetrahydrofuran, in the presence of a base (e.g., triethylamine, diisopropylethylamine, potassium carbonate, sodium carbonate, cesium carbonate, sodium hydride, potassium hydride or calcium hydride), at a temperature between 25° C. and 150° C., preferably between 50° C. and 120° C.

In certain embodiments, compounds provided herein may contain one or more asymmetric carbon atoms, so that the compound can exist in different stereoisomeric forms. Such forms can be, for example, racemates or optically active forms. As noted above, all stereoisomers are encompassed by the present invention. Nonetheless, it may be desirable in certain instances to obtain single enantiomers (i.e., optically active forms). Standard methods for preparing single enantiomers include asymmetric synthesis and resolution of the racemates. Resolution of the racemates can be accomplished, for example, by conventional methods such as crystallization in the presence of a resolving agent, or chromatography using, for example a chiral HPLC column.

Compounds may be radiolabeled by carrying out their synthesis using precursors comprising at least one atom that is a radioisotope. Each radioisotope is preferably carbon (e.g., ¹⁴C), hydrogen (e.g., ³H), sulfur (e.g., ³⁵S) or iodine (e.g., ¹²⁵I). Tritium labeled compounds may also be prepared catalytically via platinum-catalyzed exchange in tritiated acetic acid, acid-catalyzed exchange in tritiated trifluoroacetic acid, or heterogeneous-catalyzed exchange with tritium gas using the compound as substrate. In addition, certain precursors may be subjected to tritium-halogen exchange with tritium gas, tritium gas reduction of unsaturated bonds, or reduction using sodium borotritide, as appropriate. Preparation of radiolabeled compounds may be conveniently performed by a radioisotope supplier specializing in custom synthesis of radiolabeled probe compounds.

### Pharmaceutical Compositions

Compounds and pharmaceutically acceptable salts thereof provided herein can be administered as the neat chemical, but are preferably administered as a pharmaceutical composition comprising a MCH receptor modulator as described herein, together with at least one physiologically acceptable carrier, excipient, adjuvant or diluent. Pharmaceutical compositions may comprise, for example, water, buffers (e.g., neutral buffered saline or phosphate buffered saline), ethanol, mineral oil, vegetable oil, dimethylsulfoxide, carbohydrates (e.g., glucose, mannose, sucrose or dextrans), mannitol, proteins, adjuvants, polypeptides or amino acids such as glycine, antioxidants, chelating agents such as EDTA or glutathione and/or preservatives. Preferred pharmaceutical compositions are formulated for oral delivery to humans or other animals (e.g., companion animals such as dogs).

Pharmaceutical compositions provided herein may also contain additional active agents, which can be chosen from a wide variety of molecules and can function in different ways to enhance the therapeutic effects of a MCH receptor modulator, or to provide a separate therapeutic effect that does not substantially interfere with the activity of the MCH receptor modulator. Such optional active agents, when present, are typically employed in the compositions described herein at a level ranging from about 0.01% to about 50% by weight of the composition, preferably 0.1% to 25%, 0.2% to 15, 0.5% to 10% or 0.5% to 5% by weight of the composition. For example, compositions intended for the treatment of eating disorders, particularly obesity and bulimia nervosa, may further comprise leptin, a leptin receptor agonist, a melanocortin receptor 4 (MC4) agonist, sibutramine, dexfenfluramine, a growth hormone secretagogue, a beta-3 agonist, a 5HT-2 agonist, an orexin antagonist, a neuropeptide Y₁ or Y₅ antagonist, a galanin antagonist, a CCK agonist, a GLP-1 agonist, a CB1 antagonist such as Rimonabant, and/or a corticotropin-releasing hormone agonist. Other active ingredients that may be included within the compositions provided herein include antidepressants, inhibitors of dipeptidyl peptidase IV (DPP IV) and/or diuretics.

Pharmaceutical carriers must be of sufficiently high purity and sufficiently low toxicity to render them suitable for administration to the animal being treated. The carrier can be inert or it can possess pharmaceutical benefits. The amount of carrier employed in conjunction with the compound is sufficient to provide a practical quantity of material for administration per unit dose of the compound.

Exemplary pharmaceutically acceptable carriers or components thereof are sugars, such as lactose, glucose and sucrose; starches, such as corn starch and potato starch; cellulose and its derivatives, such as sodium carboxymethyl cellulose, ethyl cellulose and methyl cellulose; powdered tragacanth; malt; gelatin; talc; solid lubricants, such as stearic acid and magnesium stearate; calcium sulfate; synthetic oils; vegetable oils, such as peanut oil, cottonseed oil, sesame oil, olive oil and corn oil; polyols such as propylene glycol, glycerine, sorbitol, mannitol and polyethylene glycol; alginic acid; phosphate buffer solutions; emulsifiers, such as the TWEENS; wetting agents, such sodium lauryl sulfate; coloring agents; flavoring agents; tableting agents; stabilizers; antioxidants; preservatives; pyrogen-free water; isotonic saline; and phosphate buffer solutions.

Effective concentrations of one or more of the MCH receptor modulators provided herein are mixed with a suitable pharmaceutical carrier, excipients, adjuvant or vehicle. In instances in which the compounds exhibit insufficient solubility, methods for solubilizing compounds may be used. Such methods are known to those of skill in this art and include, but are not limited to, using cosolvents, such as dimethylsulfoxide (DMSO), using surfactants, such as TWEEN, or dissolution in aqueous sodium bicarbonate. Derivatives of the compounds, such as salts of the compounds or prodrugs of the compounds may also be used in formulating effective pharmaceutical compositions.

Upon mixing or addition of the MCH receptor modulator(s), the resulting mixture may be a solution, suspension, emulsion or the like. The form of the resulting mixture depends upon a number of factors, including the intended mode of administration and the solubility of the compound in the chosen carrier or vehicle.

MCH receptor modulators may be administered orally, topically, parenterally, by inhalation or spray, sublingually, transdermally, via buccal administration, rectally, as an ophthalmic solution, or by other means, in dosage unit formulations.

Dosage formulations suitable for oral use, include, for example, tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents, such as sweetening agents, flavoring agents, coloring agents and preserving agents, in order to provide pharmaceutically elegant and palatable preparations. Orally administered compositions also include liquid solutions, emulsions, suspensions, powders, granules, elixirs, tinctures, syrups and the like. The pharmaceutically acceptable carriers suitable for preparation of such compositions are well known in the art. Oral formulations may contain preservatives, flavoring agents, sweetening agents, such as sucrose or saccharin, taste-masking agents and coloring agents.

Typical components of carriers for syrups, elixirs, emulsions and suspensions include ethanol, glycerol, propylene glycol, polyethylene glycol, liquid sucrose, sorbitol and water. Syrups and elixirs may be formulated with sweetening agents, for example glycerol, propylene glycol, sorbitol or sucrose. Such formulations may also contain a demulcent.

### Orally Administered Liquid Formulations

MCH receptor modulators can be incorporated into oral liquid preparations such as, for example, aqueous or oily suspensions, solutions, emulsions, syrups, or elixirs. Moreover, formulations containing these compounds can be presented as a dry product for constitution with water or other suitable vehicle before use. Such liquid preparations can contain conventional additives, such as suspending agents (e.g., sorbitol syrup, methyl cellulose, glucose/sugar, syrup, gelatin, hydroxyethyl cellulose, carboxymethyl cellulose, aluminum stearate gel and hydrogenated edible fats), emulsifying agents (e.g., lecithin, sorbitan monsoleate or acacia), non-aqueous vehicles, which can include edible oils (e.g., almond oil, fractionated coconut oil, silyl esters, propylene glycol and ethyl alcohol) and preservatives (e.g., methyl or propyl p-hydroxybenzoate and sorbic acid).

### Suspensions

For a suspension, typical suspending agents include methylcellulose, sodium carboxymethyl cellulose, AVICEL RC-591, tragacanth and sodium alginate; typical wetting agents include lecithin and polysorbate 80; and typical preservatives include methyl paraben and sodium benzoate.

Aqueous suspensions contain the active material(s) in admixture with excipients suitable for the manufacture of aqueous suspensions. Such excipients are suspending agents, for example sodium carboxymethylcellulose, methylcellulose, hydropropylmethylcellulose, sodium alginate, polyvinylpyrrolidone, gum tragacanth and gum acacia; dispersing or wetting agents; may be a naturally-occurring phosphatide, for example, lecithin or condensation products of an alklylene oxide with fatty acids, for example polyoxyethylene stearate or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol substitute, or condensation products of ethylene oxide with partial esters derived from fatty acids and hexitol anhydrides, for example polyethylene sorbitan substitute. The aqueous suspensions may also contain one or more preservatives, for example ethyl or n-propyl p-hydroxybenzoate.

Oily suspensions may be formulated by suspending the active ingredients in a vegetable oil, for example peanut oil, olive oil, sesame oil or coconut oil, or in a mineral oil such as liquid paraffin. The oily suspensions may contain a thickening agent, for example beeswax, hard paraffin or cetyl alcohol. Sweetening agents, such as those set forth above, and flavoring agents may be added to provide palatable oral preparations. These compositions may be preserved by the addition of an anti-oxidant such as ascorbic acid.

### Emulsions

Pharmaceutical compositions provided herein may also be in the form of oil-in-water emulsions. The oily phase may be a vegetable oil, for example olive oil or peanut oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation products of the said partial esters with ethylene oxide, for example polyoxyethylene sorbitan monoleate.

### Dispersible Powders

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above.

### Tablets and Capsules

Tablets typically comprise conventional pharmaceutically compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint and fruit flavors, are useful adjuvants for chewable tablets. Capsules (including time release and sustained release formulations) typically comprise one or more solid diluents disclosed above. The selection of carrier components often depends on secondary considerations like taste, cost and shelf stability.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methylcellulose phthalate, ethyl cellulose, Eudragit coatings, waxes and shellac.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

### Injectable and Parenteral Formulations

Pharmaceutical compositions may be in the form of a sterile injectable aqueous or oleaginous suspension. Such a suspension may be formulated according to the known art using dispersing or wetting agents and suspending agents as described above. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid are useful in the preparation of injectables.

MCH receptor modulators may be administered parenterally in a sterile medium. Parenteral administration includes subcutaneous injections, intravenous, intramuscular, intrathecal injection or infusion techniques. The MCH receptor modulator(s), depending on the vehicle and concentration used, can either be suspended or dissolved in the vehicle. Adjuvants such as local anesthetics, preservatives and buffering agents can also be dissolved in the vehicle. In many compositions for parenteral administration, the carrier comprises at least about 90% by weight of the total composition. Preferred carriers for parenteral administration include propylene glycol, ethyl oleate, pyrrolidone, ethanol and sesame oil.

### Suppositories

MCH receptor modulators may also be administered in the form of suppositories for rectal administration of the drug. These compositions can be prepared by mixing the drug with a suitable non-irritating excipient that is solid at ordinary temperatures but liquid at rectal temperature and will therefore melt in the rectum to release the drug. Such materials include cocoa butter and polyethylene glycols.

### Topical Formulations

MCH receptor modulators may be formulated for local or topical application, such as for topical application to the skin and mucous membranes, such as in the eye, in the form of gels, creams and lotions and for application to the eye. Topical compositions of the present invention may be in any form including, for example, solutions, creams, ointments, gels, lotions, milks, cleansers, moisturizers, sprays, skin patches and the like.

Such solutions may be formulated as 0.01%-10% isotonic solutions, pH about 5-7, with appropriate salts. MCH receptor modulators may also be formulated for transdermal administration as a transdermal patch.

Topical compositions containing the active compound can be admixed with a variety of carrier materials well known in the art, such as, for example, water, alcohols, aloe vera gel, allantoin, glycerine, vitamin A and E oils, mineral oil, propylene glycol, PPG-2 myristyl propionate and the like. Other materials suitable for use in topical carriers include, for example, emollients, solvents, humectants, thickeners and powders. Examples of each of these types of materials, which can be used singly or as mixtures of one or more materials, are as follows: emollients, such as stearyl alcohol, glyceryl monoricinoleate, glyceryl monostearate, propane-1,2-diol, butane-1,3-diol, mink oil, cetyl alcohol, iso-propyl isostearate, stearic acid, iso-butyl palmitate, isocetyl stearate, oleyl alcohol, isopropyl laurate, hexyl laurate, decyl oleate, octadecan-2-ol, isocetyl alcohol, cetyl palmitate, dimethylpolysiloxane, di-n-butyl sebacate, isopropyl myristate, iso-propyl palmitate, iso-propyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate and myristyl myristate; propellants, such as propane, butane, iso-butane, dimethyl ether, carbon dioxide and nitrous oxide; solvents, such as ethyl alcohol, methylene chloride, iso-propanol, castor oil, ethylene glycol monoethyl ether, diethylene glycol monobutyl ether, diethylene glycol monoethyl ether, dimethyl sulphoxide, dimethyl formamide, tetrahydrofuran; humectants, such as glycerin, sorbitol, sodium 2-pyrrolidone-5-carboxylate, soluble collagen, dibutyl phthalate and gelatin; and powders, such as chalk, talc, fullers earth, kaolin, starch, gums, colloidal silicon dioxide, sodium polyacrylate, tetra alkyl ammonium smectites, trialkyl aryl ammonium smectites, chemically modified magnesium aluminium silicate, organically modified montmorillonite clay, hydrated aluminium silicate, fumed silica, carboxyvinyl polymer, sodium carboxymethyl cellulose and ethylene glycol monostearate.

MCH receptor modulators may also be topically administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

### Other Formulations

Other compositions useful for attaining systemic delivery of the subject compounds include sublingual, buccal and nasal dosage forms. Such compositions typically comprise one or more of soluble filler substances such as sucrose, sorbitol and mannitol, and binders such as acacia, microcrystalline cellulose, carboxymethyl cellulose and hydroxypropyl methylcellulose. Glidants, lubricants, sweeteners, colorants, antioxidants and flavoring agents disclosed above may also be included.

Compositions for inhalation typically can be provided in the form of a solution, suspension or emulsion that can be administered as a dry powder or in the form of an aerosol using a conventional propellant (e.g., dichlorodifluoromethane or trichlorofluoromethane).

In addition to or together with the above modes of administration, a modulator may be conveniently added to food or drinking water (e.g., for administration to non-human animals including companion animals, such as dogs and cats and livestock). Animal feed and drinking water compositions may be formulated so that the animal takes in an appropriate quantity of the composition along with its diet. It may also be convenient to present the composition as a premix for addition to feed or drinking water.

### Additional Components

The compositions of the present invention may also optionally comprise an activity enhancer. The activity enhancer can be chosen from a wide variety of molecules that function in different ways to enhance MCH receptor modulator effect. Particular classes of activity enhancers include skin penetration enhancers and absorption enhancers.

### Packaged Formulations

Pharmaceutical compositions may be packaged for treating or preventing a disease or disorder that is associated with MCH receptor activation (e.g., treatment of metabolic disorders such as diabetes, heart disease, stroke, eating disorders such as obesity or bulimia, skin disorders such as vitiligo, or sexual disorders such as anorgasmic or psychogenic impotence), or for promoting weight loss. Other such diseases and disorders are described herein. Packaged pharmaceutical compositions include a container holding a therapeutically effective amount of MCH receptor modulator as described herein and instructions (e.g., labeling) indicating that the contained composition is to be used for treating or preventing a disease or disorder that is associated with MCH receptor activation in the patient. Prescribing information may be provided to a patient or health care provider or as a label in a packaged pharmaceutical formulation. Prescribing information may include, for example, efficacy, dosage and administration, contraindication and adverse reaction information pertaining to the pharmaceutical formulation.

### Dosages

Modulators are generally present within a pharmaceutical composition in a therapeutically effective amount. Compositions providing dosage levels ranging from about 0.1 mg to about 140 mg per kilogram of body weight per day are preferred (about 0.5 mg to about 7 g per human patient per day), with dosages ranging from 0.1 mg to 50 mg, 30 mg or 10 mg particularly preferred. The amount of active ingredient that may be combined with the carrier materials to produce a single dosage form will vary depending upon the host treated and the particular mode of administration. Dosage unit forms will generally contain between from about 1 mg to about 500 mg of an active ingredient. It will be understood, however, that the optimal dose for any particular patient will depend upon a variety of factors, including the activity of the specific compound employed; the age, body weight, general health, sex and diet of the patient; the time and route of administration; the rate of excretion; any simultaneous treatment, such as a drug combination; and the type and severity of the particular disease undergoing treatment. Dosage units will generally contain between from about 10 μg to about 500 mg of an active ingredient. Optimal dosages may be established using routine testing and procedures that are well known in the art.

### Methods of Use

Within certain aspects, the present invention provides methods for inhibiting the development of a disease or disorder responsive to MCH receptor modulation. In other words, therapeutic methods provided herein may be used to treat a patient already afflicted with a disease, or may be used to prevent or delay the onset of such a disease in a patient who is free of detectable disease that is associated with MCH receptor activation. As noted above, a disease or disorder is “associated with MCH receptor activation” if it is characterized by inappropriate stimulation of MCH receptor, regardless of the amount of MCH present locally, and/or is responsive to modulation of MCH receptor activity. Such conditions include, for example, metabolic disorders (such as diabetes), heart disease, stroke, eating disorders (such as obesity and bulimia nervosa), disorders of the skin such as vitiligo, and sexual disorders such as anorgasmic or psychogenic impotence. These conditions may be diagnosed and monitored using criteria that have been established in the art. In addition, MCH antagonists provided herein may be used to promote weight loss in patients, and MCH agonists provided herein may be used to promote weight gain in patients. Patients may include humans, domesticated companion animals (pets, such as dogs) and livestock animals, with dosages and treatment regimes as described above.

Additional conditions that are associated with MCH receptor activation include:
Cognitive impairment and memory disorders, such as Alzheimer's disease, Parkinson's disease, mild cognitive impairment (MC1), age-related cognitive decline (ARCD), stroke, traumatic brain injury, AIDS associated dementia, and dementia associated with depression, anxiety and psychosis (including schizophrenia and hallucinatory disorders);
Anxiety, depression and other mood disorders, including general anxiety disorder (GAD), agoraphobia, panic disorder with and without agoraphobia, social phobia, specific phobia, post traumatic stress disorder, obsessive compulsive disorder (OCD), dysthymia, adjustment disorders with disturbance of mood and anxiety, separation anxiety disorder, anticipatory anxiety acute stress disorder, adjustment disorders, cyclothymia;
Reward system disorders such as addiction (e.g., opioid, nicotine or alcohol);
Pain such as migraine, peripheral inflammatory pain, neuropathic pain and sympathetic nervous system associated pain; and
Peripheral indications such as respiratory disorders (e.g., asthma), urinary disorders (e.g., urinary incontinence), gastrointestinal disorders, reproductive function disorders and cardiovascular disorders (e.g., arteriosclerosis and hypertension).

Frequency of dosage may vary depending on the compound used and the particular disease to be treated or prevented. In general, for treatment of most disorders, a dosage regimen of 4 times daily or less is preferred. For the treatment of eating disorders, including obesity, a dosage regimen of 1 or 2 times daily is particularly preferred. For the treatment of impotence a single dose that rapidly reaches effective concentrations is desirable. It will be understood, however, that the specific dose level for any particular patient will depend upon a variety of factors including the activity of the specific compound employed, the age, body weight, general health, sex, diet, time of administration, route of administration, rate of excretion, drug combination and the severity of the particular disease undergoing therapy. In certain embodiments, administration at meal times is preferred. In general, the use of the minimum dosage that is sufficient to provide effective therapy is preferred. Patients may generally be monitored for therapeutic effectiveness using assays suitable for the condition being treated or prevented, which will be familiar to those of ordinary skill in the art.

Within separate aspects, the present invention provides a variety of in vitro uses for the compounds provided herein. For example, such compounds may be used as probes for the detection and localization of MCH receptors, in samples such as tissue sections, as positive controls in assays for receptor activity, as standards and reagents for determining the ability of a candidate agent to bind to MCH receptor, or as radiotracers for positron emission tomography (PET) imaging or for single photon emission computerized tomography (SPECT). Such assays can be used to characterize MCH receptors in living subjects. Such compounds are also useful as standards and reagents in determining the ability of a potential pharmaceutical to bind to melanin concentrating hormone receptor.

Within methods for determining the presence or absence of MCH receptor in a sample, a sample may be incubated with a compound as provided herein under conditions that permit binding of the compound to MCH receptor. The amount of compound bound to MCH receptor in the sample is then detected. For example, a compound may be labeled using any of a variety of well known techniques (e.g., radiolabeled with a radionuclide such as tritium, as described herein), and incubated with the sample (which may be, for example, a preparation of cultured cells, a tissue preparation or a fraction thereof). A suitable incubation time may generally be determined by assaying the level of binding that occurs over a period of time. Following incubation, unbound compound is removed, and bound compound detected using any method for the label employed (e.g., autoradiography or scintillation counting for radiolabeled compounds; spectroscopic methods may be used to detect luminescent groups and fluorescent groups). As a control, a matched sample may be simultaneously contacted with radiolabeled compound and a greater amount of unlabeled compound. Unbound labeled and unlabeled compound is then removed in the same fashion, and bound label is detected. A greater amount of detectable label in the test sample than in the control indicates the presence of MCH receptor in the sample. Detection assays, including receptor autoradiography (receptor mapping) of MCH receptors in cultured cells or tissue samples may be performed as described by Kuhar in sections 8.1.1 to 8.1.9 of Current Protocols in Pharmacology (1998) John Wiley & Sons, New York.

Modulators provided herein may also be used within a variety of well known cell culture and cell separation methods. For example, modulators may be linked to the interior surface of a tissue culture plate or other cell culture support, for use in immobilizing MCH receptor-expressing cells for screens, assays and growth in culture. Such linkage may be performed by any suitable technique, such as the methods described above, as well as other standard techniques. Modulators may also be used to facilitate cell identification and sorting in vitro, permitting the selection of cells expressing a MCH receptor. Preferably, the modulator(s) for use in such methods are labeled as described herein. Within one preferred embodiment, a modulator linked to a fluorescent marker, such as fluorescein, is contacted with the cells, which are then analyzed by fluorescence activated cell sorting (FACS).

Within other aspects, methods are provided for modulating binding of MCH to an MCH receptor in vitro or in vivo, comprising contacting a MCH receptor with a sufficient amount of a modulator provided herein, under conditions suitable for binding of MCH to the receptor. Preferably, within such methods, MCH binding to receptor is inhibited by the modulator. The MCH receptor may be present in solution, in a cultured or isolated cell preparation or within a patient. Preferably, the MCH receptor is a MCHR1 receptor present in the hypothalamus. In general, the amount of compound contacted with the receptor should be sufficient to modulate MCH binding to MCH receptor in vitro within, for example, a binding assay as described in Example 2. MCH receptor preparations used to determine in vitro binding may be obtained from a variety of sources, such as from HEK 293 cells or Chinese Hamster Ovary (CHO) cells transfected with a MCH receptor expression vector, as described herein.

Also provided herein are methods for modulating the signal-transducing activity of cellular MCH receptors, by contacting MCH receptor, either in vitro or in vivo, with a sufficient amount of a modulator as described above, under conditions suitable for binding of MCH to the receptor. Preferably, within such methods, signal-transducing activity is inhibited by the modulator. The MCH receptor may be present in solution, in a cultured or isolated cell preparation or within a patient. In general, the amount of modulator contacted with the receptor should be sufficient to modulate MCH receptor signal transducing activity in vitro within, for example, a calcium mobilization assay as described in Example 3. An effect on signal-transducing activity may be assessed as an alteration in the electrophysiology of the cells, using standard techniques, such as intracellular patch clamp recording or patch clamp recording. If the receptor is present in an animal, an alteration in the electrophysiology of the cell may be detected as a change in the animal's feeding behavior.

The following Examples are offered by way of illustration and not by way of limitation. Unless otherwise specified all reagents and solvent are of standard commercial grade and are used without further purification.

### EXAMPLES

### Example 1

### Preparation of Representative Substituted Tetrahydroisoquinoline Analogues

This Example illustrates the synthesis of representative compounds of Formula I. It will be apparent that, through variation of starting compounds, these methods may be used to prepare a wide variety of such compounds. Where indicated, ¹H NMR spectra were recorded on a Varian Gemini 300 operating at 300 MHz. Data are expressed in parts-per-million (ppm) in δ units relative to tetramethylsilane (TMS, δ=0 ppm) as an internal standard.

Mass spectra (MS) are collected using electrospray MS, obtained in positive ion mode using a Waters ZMD II Mass Spectrometer. MS conditions are as follows:
Capillary voltage: 3.5 kV
Cone voltage: 30 V
Desolvation and source temperature: 250° C. and 120° C. respectively
Mass range: 100-750
Scan time: 0.5 second
Inter scan delay: 0.1 minute

HPLC analyses are performed using a Waters 600 series pump (Waters Corporation, Milford, Mass.), a Waters 996 Diode Array Detector and a Gilson 215 autosampler (Gilson Inc, Middleton, Wis.). Data are acquired using MassLynx 4.0 software, with OpenLynx processing. HPLC conditions are as follows:
Column: 4.6×50 mm XTerra MS C18, 5 μm column (Waters Milford)
Detection: UV 10 spectra/sec, 220 and 254 nm
Flow rate 4.0 mL/min
Injection volume 1-10 μl
Mobile phase A: 95% aqueous 10 mM ammonium formate, 5% methanol
Mobile phase B: 95% methanol, 5% water with 0.025% formic acid
Gradient:

| Time (min) | % B |
|---|---|
| 0 | 10 |
| 0.01 | 10 |
| 2.0 | 100 |
| 3.50 | 100 |
| 3.51 | 10 |

### Compound 1: 2-[3-(4-tert-Butyl-phenoxy)-benzyl]-6,7-dimethoxy-1,2,3,4-tetrahydroisoquinoline

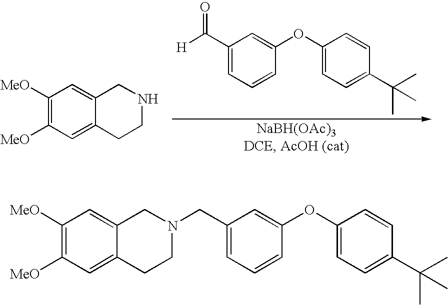

6,7-Dimethoxy-1,2,3,4-tetrahydroisoquinoline (84 mg, 0.44 mmol) and 3-(4-tert-butylphenoxy)benzaldehyde (113 μL, 0.44 mmol) are dissolved in 2 mL of anhydrous 1,2-dichloroethane under a N₂ atmosphere. Solid NaBH(OAc)₃ (129 mg, 0.61 mmol) is added, followed by addition of acetic acid (100 μL). The resulting suspension is stirred for 16 hours at room temperature. The reaction mixture is taken to basic pH by addition of aqueous NaOH (2 mL, 1M). The product is extracted with CH₂Cl₂ (3×2 mL) and the combined organic fractions washed with brine, dried over anhydrous Na₂SO₄ and concentrated under reduced pressure. The crude product is dissolved in a minimal amount of methanol and loaded onto an SCX SPE cartridge (Applied Separations). Non-basic impurities are eluted with methanol (4 mL). The desired product is collected with NH₃ (1M in methanol, 4 mL). Solvent removal under reduced pressure affords the title compound as a colorless oil. H-1 NMR (CDCl₃): 7.34 (2H, d), 7.30 (1H, d), 7.12 (1H, d), 7.08 (1H, d), 6.94 (2H, d), 6.94 (1H, t), 6.58 (1H, s), 6.48 (1H, s), 3.84 (3H, s), 3.82 (3H, s), 3.72 (2H, s), 3.62 (2H, s), 2.80 (4H, m); LC/MS m/z: (M+H)⁺ calcd for C₂₈H₃₃NO₃ 432, found 432.

### Compound 2: 2-[3-(4-tert-Butyl-phenoxy)-benzyl]-6,7-dimethoxy-1-methyl-1,2,3,4-tetrahydroisoquinoline

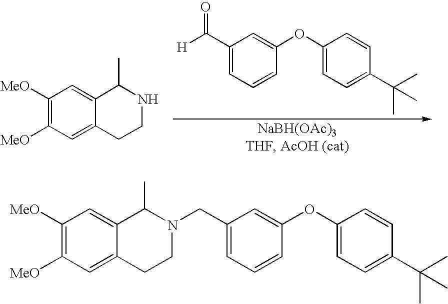

6,7-Dimethoxy-1-methyl-1,2,3,4-tetrahydroisoquinoline (63 mg, 0.30 mmol) and 3-(4-tert-butylphenoxy)benzaldehyde (76 mg, 0.30 mmol) are dissolved in 3 mL of anhydrous THF under a N₂ atmosphere. Solid NaBH(OAc)₃ (89 mg, 0.42 mmol) is added, followed by addition of acetic acid (100 μL). The reaction is stirred for 16 hours at room temperature. The resulting reaction mixture is taken to basic pH by addition of aqueous NaOH (2 mL, 1M). The product is extracted with CH₂Cl₂ (3×2 mL). The combined extracts are dried over anhydrous Na₂SO₄ and concentrated under reduced pressure. The crude residue is purified with silica gel chromatography eluting with EtOAc/hexanes (3:1) to afford the title compound as a colorless oil. H-1 NMR (CDCl₃): 7.32 (3H, m), 7.12 (1H, s), 7.10 (1H, m), 6.92 (2H, d), 6.88 (1H, m), 6.54 (1H, s), 6.50 (1H, s), 3.84 (3H, s), 3.82 (3H, s), 3.80 (2H, d), 3.72 (1H, q), 3.00-3.12 (1H, m), 2.66-2.78 (2H, m), 2.52-2.62 (1H, m), 1.34 (3H, d), 1.32 (9H, s). LC/MS m/z: (M+H)⁺ calcd for C₂₉H₃₅NO₃ 446, found 446.

### Compound 3: 6,7-dimethoxy-2-(3-p-tolyloxy-benzyl)-1,2,3,4-tetrahydroisoquinoline

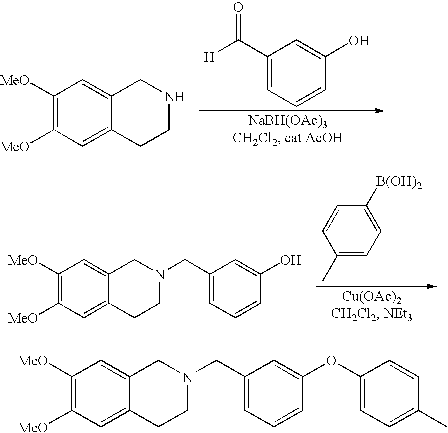

### Step 1: 3-(6,7-Dimethoxy-3,4-dihydro-1H-isoquinolin-2-ylmethyl)-phenol

6,7-Dimethoxy-1,2,3,4-tetrahydroisoquinoline (5.0 g, 25.9 mmol) and 3-hydroxybenzaldehyde (3.16 g, 25.9 mmol) are dissolved in 100 mL of anhydrous DCE under N₂ atmosphere. Solid NaBH(OAc)₃ (8.22 g, 38.8 mmol) is added, followed by addition of acetic acid (5 mL). The reaction is stirred for 16 hours at room temperature. The resulting reaction mixture is taken to basic pH by addition of aqueous NaOH (200 mL, 1M). A solid precipitates from solution and is subsequently filtered. The precipitate is washed with H₂O (50 mL) and then EtOAc (100 mL) to afford the desired product as a white solid. H-1 NMR (DMSO-d₆): 7.08 (1H, t), 6.52-6.74 (5H, m), 3.68 (3H, s), 3.64 (3H, s), 3.48 (2H, s), 3.36 (2H, s), 2.44-2.72 (4H, m). LC/MS m/z: (M+H)⁺ calcd for C₁₈H₂₁NO₃ 300, found 300.

### Step 2: 6,7-Dimethoxy-2-(3-p-tolyloxy-benzyl)-1,2,3,4-tetrahydroisoquinoline

The phenol obtained in step 1 (50 mg, 0.17 mmol) is dissolved in CH₂Cl₂ (1 mL). 4-Methylphenylboronic acid (69 mg, 0.51 mmol) is then added, followed by Cu(OAc)₂ (31 mg, 0.17 mmol) and NEt₃ (0.12 mL, 0.85 mmol). The mixture is stirred for 48 hours at room temperature. The heterogeneous mixture is filtered and the product is purified by flash chromatography over silicagel eluting with EtOAc/hexanes (3:1). The desired product is obtained as a white solid. H-1 NMR (CDCl₃): 7.26 (1H, t), 7.10-7.16 (3H, m), 7.06 (1H, s), 6.84-6.94 (3H, m), 6.58 (1H, s), 6.48 (1H, s), 3.82 (3H, s), 3.80 (3H, s), 3.64 (2H, s), 3.54 (2H, s), 2.80 (2H, m), 2.72 (2H, m), 2.32 (3H, s). LC/MS m/z: (M+H)⁺ calcd for C₂₅H₂₇NO₃ 390, found 390.

### Compound 4: 2-[3-(4-tert-butyl-phenoxy)-2-methyl-benzyl]-6,7-dimethoxy-1,2,3,4-tetrahydroisoquinoline

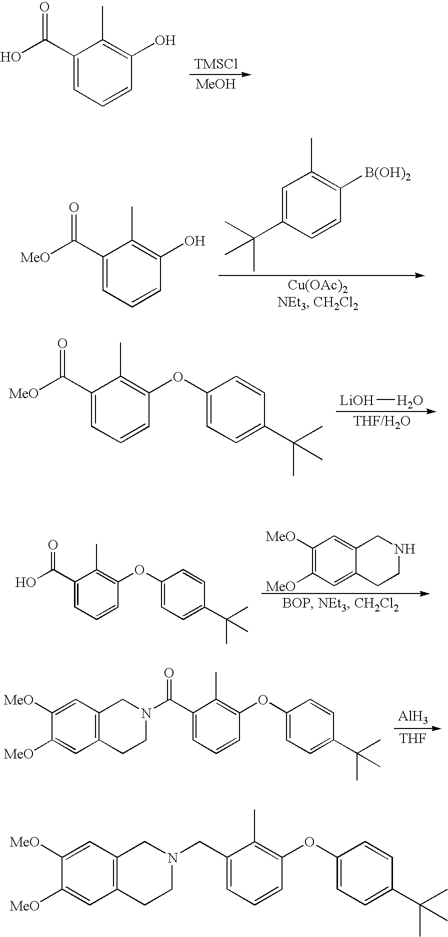

### Step 1: 3-Hydroxy-2-methylbenzoic acid methyl ester

3-Hydroxy-2-methylbenzoic acid (1.0 g, 6.58 mmol) is dissolved in 40 mL of MeOH. Chlorotrimethylsilane (2.5 mL, 19.7 mmol) is added and the resulting solution is stirred for 16 hours at room temperature. The reaction mixture is concentrated under reduced pressure and the resulting residue is purified by silicagel chromatography eluting with EtOAc/hexanes (1:1). The title compound is obtained as a pale yellow solid. LC/MS m/z: (M+H)⁺ calcd for C₉H₁₀O₃ 167, found 167.

### Step 2: 3-(4-tert-Butyl-phenoxy)-2-methyl-benzoic acid methyl ester

The 3-hydroxy-2-methylbenzoic acid methyl ester obtained in step 1 (0.20 g, 1.2 mmol) is dissolved in 5 mL of CH₂Cl₂ over powdered 4 A molecular sieves. 4-tert-Butylphenylboronic acid (0.64 g, 3.6 mmol) is then added, followed by Cu(OAc)₂ (0.22 g, 1.2 mmol) and NEt₃ (0.84 mL, 6.0 mmol). The reaction mixture is stirred for 48 hours at room temperature. The heterogeneous mixture is filtered and the product is purified by flash chromatography over silicagel eluting with EtOAc/hexanes (1:3). The title compound is isolated as pale orange oil. H-1 NMR (CDCl₃): 7.62 (1H, d), 7.30 (2H, d), 7.18 (1H, t), 7.06 (1H, d), 6.82 (2H, d), 3.92 (3H, s), 2.48 (3H, s), 1.30 (9H, s). LC/MS m/z: (M+H)⁺ calcd for C₁₉H₂₂O₃ 299, found 299.

### Step 3: 3-(4-tert-Butyl-phenoxy)-2-methyl-benzoic acid

The methyl ester obtained in step 2 (310 mg, 1.04 mmol) is dissolved in 5 mL of H₂O/THF (1:4). LiOH.H₂O is added and the reaction mixture heated at 50° C. for 16 hours. The cooled reaction mixture is acidified to pH 4 with 1N HCl. The product is extracted with CH₂Cl₂ (2×25 mL). Concentration under reduced pressure afforded an off-white solid. H-1 NMR (DMSO-d₆): 7.58 (1H, d), 7.34 (2H, d), 7.26 (1H, t), 7.06 (1H, d), 6.80 (2H, d), 2.32 (3H, s), 1.26 (9H, s).

### Step 4: [3-(4-tert-Butyl-phenoxy)-2-methyl-phenyl]-(6,7-dimethoxy-3,4-dihydro-1H-isoquinolin-2-yl)-methanone

6,7-Dimethoxy-1,2,3,4-tetrahydroisoquinoline (166 mg, 0.86 mmol) and 3-(4-tert-butylphenoxy)-2-methylbenzoic acid (244 mg, 0.86 mmol) are dissolved in 5 mL of CH₂Cl₂. NEt₃ (0.18 mL, 1.29 mmol) and BOP (0.57 g, 1.29 mmol) are added and the reaction mixture is stirred at room temperature for 16 hours. The reaction mixture is concentrated in vacuo and the resulting residue dissolved in EtOAc and washed with 1N NaOH (3×10 mL). The combined organic extracts are dried over anhydrous Na₂SO₄ and the solvent evaporated under reduced pressure. Flash chromatography on silicagel eluting with EtOAc/hexanes (1:1) yields the desired product as a white solid. LCMS m/z: (M+H)⁺ calcd for C₂₉H₃₃NO₄ 460, found 460.

### Step 5: 2-[3-(4-tert-Butyl-phenoxy)-2-methyl-benzyl]-6,7-dimethoxy-1,2,3,4-tetrahydroisoquinoline

The amide obtained in step 4 (361 mg, 0.79 mmol) is dissolved in 5 mL of anhydrous THF. Alane-N,N-dimethylethylamine complex (3.95 mL, 0.5 M solution in toluene, Aldrich Chemical Co.) is added and the mixture is stirred at room temperature for 5 hours. The reaction is quenched by dropwise addition of EtOAc/MeOH (1:1, 1 mL) until all bubbling has ceased. The resulting slurry is filtered through a silicagel plug. The product is eluted with EtOAc and concentrated to afford a colorless oil. H-1 NMR (CDCl₃): 7.30 (2H, d), 7.08-7.18 (2H, m), 6.82 (3H, m), 6.60 (1H, s), 6.50 (1H, s), 3.82 (3H, s), 3.80 (3H, s), 3.68 (2H, s), 3.58 (2H, s), 2.72-2.84 (4H, m), 2.28 (3H, s), 1.30 (9H, s). LC/MS m/z: (M+H)⁺ calcd for C₂₉H₃₅NO₃ 446, found 446.

### Compound 5: 2-[2-(4-tert-Butyl-phenoxy)-pyridin-4-ylmethyl]-6,7-dimethoxy-1,2,3,4-tetrahydroisoquinoline

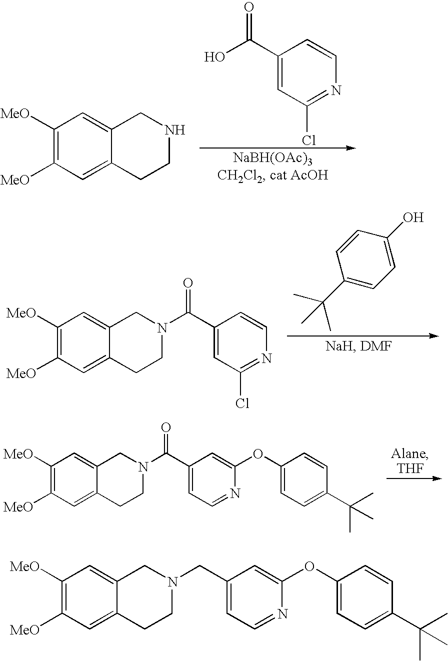

### Step 1: (2-Chloro-pyridin-4-yl)-(6,7-dimethoxy-3,4-dihydro-1H-isoquinolin-2-yl)-methanone

6,7-Dimethoxy-1,2,3,4-tetrahydroisoquinoline hydrochloride (1.00 g, 4.35 mmol) and 2-chloroisonicotinic acid (0.69 g, 4.35 mmol) are dissolved in 20 mL of CH₂Cl₂. NEt₃ (1.21 mL, 8.70 mmol) and BOP (3.85 g, 8.70 mmol) are added and the reaction is stirred at room temperature for 16 h. The mixture is concentrated in vacuo and the resulting residue is dissolved in EtOAc and washed with 1N NaOH (3×20 mL). The combined organic extracts are dried over anhydrous Na₂SO₄ and concentrated. The product is purified by silicagel chromatography (EtOAc/hexanes, 1:1). The title compound is obtained as a white crystalline solid. H-1 NMR (CDCl₃): 8.50 (1H, d), 7.36 (1H, s), 7.26 (1H, m), 6.64 (2H, d), 4.80 (1H, s), 4.42 (1H, s), 3.96 (1H, t), 3.88 (6H, s), 3.56 (1H, t), 2.90 (1H, t), 2.80 (1H, t). LC/MS m/z: (M+H)⁺ calcd for C₁₇H₁₇ClN₂O₃ 333, found 333.

### Step 2: [2-(4-tert-Butyl-phenoxy)-pyridin-4-yl]-(6,7-dimethoxy-3,4-dihydro-1H-isoquinolin-2-yl)-methanone

tert-Butylphenol (0.61 g, 4.03 mmol) is dissolved in 20 mL of anhydrous DMF. Sodium hydride (0.24 g, 6.05 mmol, 60% dispersion in mineral oil) is added and the reaction stirred at room temperature for 15 minutes. The chloropyridine obtained in step 1 (1.34 g, 4.03 mmol) is added and the reaction heated at 110° C. for 16 hours. The reaction is cooled to room temperature and the mixture is partitioned between EtOAc (20 mL) and NaOH (20 mL, 1M). The organic extract is washed with saturated brine (3×20 mL) followed by 1N HCl (2×20 mL), 1N NaOH and brine until neutrality, dried over anhydrous Na₂SO₄ and concentrated under reduced pressure. The resulting residue is triturated with ethyl ether and collected by filtration to afford the desired compound as a pale pink solid. H-1 NMR (DMSO-d₆): 8.22 (1H, d), 7.38-7.44 (2H, m), 6.94-7.18 (4H, m), 6.68-6.86 (2H, m), 4.66 (1H, s), 4.42 (1H, s), 3.68-3.80 (7H, m), 3.48 (1H, t), 2.68-2.82 (2H, m), 1.30 (9H, s). LC/MS m/z: (M+H)⁺ calcd for C₂₇H₃₀N₂O₄ 447, found 447.

### Step 3: 2-[2-(4-tert-Butyl-phenoxy)-pyridin-4-ylmethyl]-6,7-dimethoxy-1,2,3,4-tetrahydroisoquinoline

The amide obtained in step 2 (0.63 g, 1.41 mmol) is dissolved in 10 mL of anhydrous THF. Alane-N,N-dimethylethylamine complex (7.1 mL, 0.5 M solution in toluene) is added and the mixture is stirred at room temperature for 3 hours. The reaction is quenched by dropwise addition of EtOAc/MeOH (1:1, 5 mL) until all bubbling ceases. The resulting slurry is filtered through a celite plug and rinsed with EtOAc (75 mL). The filtrate is concentrated under reduced pressure to afford the title compound as a clear, colorless oil. ¹H NMR (CDCl₃): 8.12 (1H, d), 7.40 (2H, d), 7.06 (2H, d), 7.04 (1H, d), 6.94 (1H, s), 6.60 (1H, s), 6.50 (1H, s), 3.84 (3H, s), 3.82 (3H, s), 3.68 (2H, s), 3.58 (2H, s), 2.82 (2H, t), 2.72 (2H, t), 1.32 (9H, s). LC/MS m/z: (M+H)⁺ calcd for C₂₇H₃₂N₂O₃ 433, found 433.

Additional representative compounds are shown in the following table, in which a “*” in the column labeled “Kᵢ” indicates that the Kᵢ determined as described in Example 2 is less than 1 micromolar. The Kᵢ for compounds 1, 3 and 5, above, is also less than 1 micromolar. Mass spectroscopy data in the “MS” column is observed data obtained as described above and is presented as (M+1).

| Compound | | Name | NMR | MS | Kᵢ |
|---|---|---|---|---|---|
| 6. | 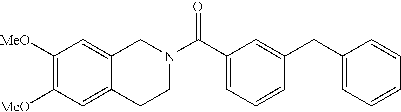 | (3-Benzyl-phenyl)-(6,7-dimethoxy-3,4-dihydro-1H-isoquinolin-2-yl)-methanone | | 388 | |
| 7. | 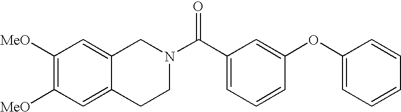 | (6,7-Dimethoxy-3,4-dihydro-1H-isoquinolin-2-yl)-(3-phenoxy-phenyl)-methanone | | 390 | |
| 8. | 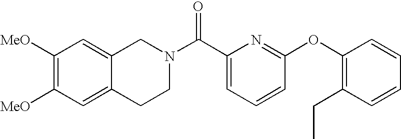 | (6,7-Dimethoxy-3,4-dihydro-1H-isoquinolin-2-yl)-[6-(2-ethyl-phenoxy)-pyridin-2-yl]-methanone | | 419 | |
| 9. | 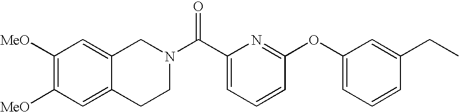 | (6,7-Dimethoxy-3,4-dihydro-1H-isoquinolin-2-yl)-[6-(3-ethyl-phenoxy)-pyridin-2-yl]-methanone | | 419 | |
| 10. | 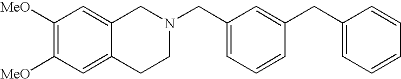 | 2-(3-Benzyl-benzyl)-6,7-dimethoxy-1,2,3,4-tetrahydroisoquinoline | | 374 | * |
| 11. | 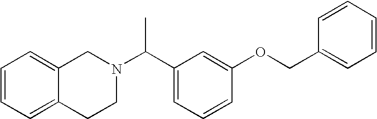 | 2-[1-(3-Benzyloxy-phenyl)-ethyl]-1,2,3,4-tetrahydroisoquinoline | | 344 | |
| 12. | 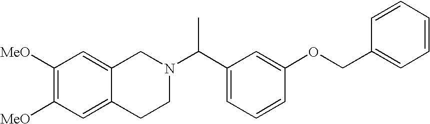 | 2-[1-(3-Benzyloxy-phenyl)-ethyl]-6,7-dimethoxy-1,2,3,4-tetrahydroisoquinoline | | 404 | |
| 13. | 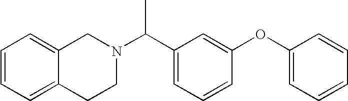 | 2-[1-(3-Phenoxy-phenyl)-ethyl]-1,2,3,4-tetrahydroisoquinoline | | 330 | |
| 14. | 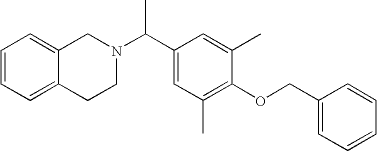 | 2-[1-(4-Benzyloxy-3,5-dimethyl-phenyl)-ethyl]-1,2,3,4-tetrahydroisoquinoline | | 372 | |
| 15. | 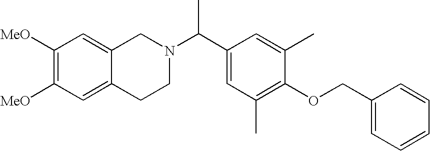 | 2-[1-(4-Benzyloxy-3,5-dimethyl-phenyl)-ethyl]-6,7-dimethoxy-1,2,3,4-tetrahydroisoquinoline | | 432 | |
| 16. | 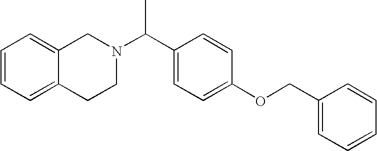 | 2-[1-(4-Benzyloxy-phenyl)-ethyl]-1,2,3,4-tetrahydroisoquinoline | | 344 | |
| 17. | 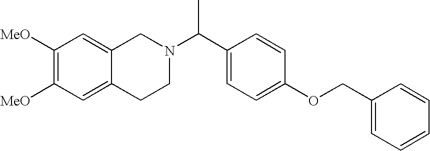 | 2-[1-(4-Benzyloxy-phenyl)-ethyl]-6,7-dimethoxy-1,2,3,4-tetrahydroisoquinoline | | 404 | |
| 18. | 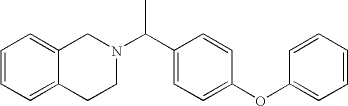 | 2-[1-(4-Phenoxy-phenyl)-ethyl]-1,2,3,4-tetrahydroisoquinoline | | 330 | |
| 19. | 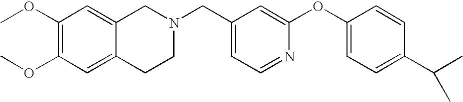 | 2-[2-(4-Isopropyl-phenoxy)-pyridin-4-ylmethyl]-6,7-dimethoxy-1,2,3,4-tetrahydroisoquinoline | | 419 | * |
| 20. | 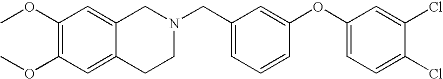 | 2-[3-(3,4-Dichloro-phenoxy)-benzyl]-6,7-dimethoxy-1,2,3,4-tetrahydroisoquinoline | | 444 | |
| 21. | 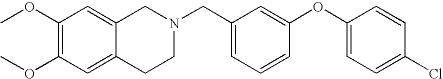 | 2-[3-(4-Chloro-phenoxy)-benzyl]-6,7-dimethoxy-1,2,3,4-tetrahydroisoquinoline | 7.40(2 H, d), 7.36(1 H, t), 7.12(1 H, d), 7.08-6.98(3 H, m), 6.92(1 H, d), 6.64(1 H, s), 6.56(1 H, s),3.68(3 H, s), 3.66(3 H, s), 3.60(2 H, s), 3.42(2 H, s),2.72-2.58(4 H, m) | 410 | * |
| 22. | 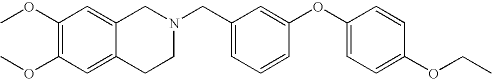 | 2-[3-(4-Ethoxy-phenoxy)-benzyl]-6,7-dimethoxy-1,2,3,4-tetrahydroisoquinoline | | 420 | * |
| 23. | 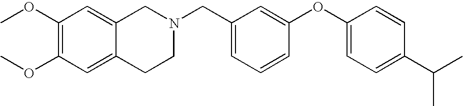 | 2-[3-(4-Isopropyl-phenoxy)-benzyl]-6,7-dimethoxy-1,2,3,4-tetrahydroisoquinoline | | 418 | * |
| 24. | 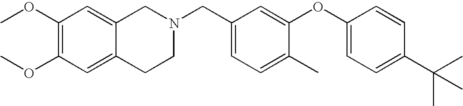 | 2-[3-(4-tert-Butyl-phenoxy)-4-methyl-benzyl]-6,7-dimethoxy-1,2,3,4-tetrahydroisoquinoline | 7.30(2 H, d), 7.20(1 H, m), 7.10(1 H, d), 6.96(1 H,s), 6.82(2 H, d), 6.58(1 H, s), 6.48(1 H, s), 3.82(3 H, s),3.80(3 H, s), 3.60(2 H, s), 3.52(2 H, s), 2.76(2 H, t),2.66(2 H, t), 2.22(3 H, s), 1.32(9 H, s) | 446 | |
| 25. | 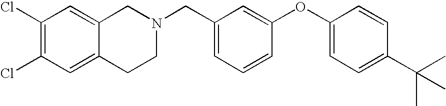 | 2-[3-(4-tert-Butyl-phenoxy)-benzyl]-6,7-dichloro-1,2,3,4-tetrahydroisoquinoline | 7.38-7.28(5 H, m), 7.06(1 H, d), 6.96-6.84(4 H, m),3.62(2 H, s), 3.50(2 H, s), 2.74(2 H, t), 2.60(2 H, t),1.22(9 H, s) | 440 | |
| 26. | 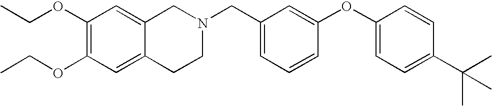 | 2-[3-(4-tert-Butyl-phenoxy)-benzyl]-6,7-diethoxy-1,2,3,4-tetrahydroisoquinoline | 7.36-7.26(3 H, m), 7.12(1 H, d), 7.08(1 H, s), 6.96-6.88(3 H, m), 6.60(1 H, s), 6.50(1 H, s), 4.04(2 H, q),4.00(2 H, q), 3.64(2 H, s), 3.54(2 H, s), 2.76(2 H, m),2.70(2 H, m), 1.42(3 H, t), 1.40(3 H, t), 1.32(9 H, s) | 460 | * |
| 27. | 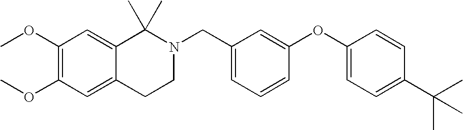 | 2-[3-(4-tert-Butyl-phenoxy)-benzyl]-6,7-dimethoxy-1,1-dimethyl-1,2,3,4-tetrahydroisoquinoline | 7.34(2 H, d), 7.28(1 H, t), 7.16(1 H, d), 7.12(1 H, s),6.94(2 H, d), 6.88(1 H, d), 6.74(1 H, s), 6.50(1 H, s),3.88(3 H, s), 3.84(3 H, s), 3.70(2 H, s), 2.72-2.62(4 H, m), 1.46(6 H, s), 1.30(9 H, s) | 460 | |
| 28. | 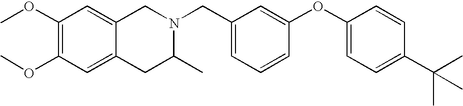 | 2-[3-(4-tert-Butyl-phenoxy)-benzyl]-6,7-dimethoxy-3-methyl-1,2,3,4-tetrahydroisoquinoline | 7.32(2 H, d), 7.26(1 H, t), 7.10(1 H, d), 7.08(1 H, s),6.94(2 H, d), 6.88(1 H, d), 6.58(1 H, s), 6.46(1 H, s),3.84(3 H, s), 3.80(3 H, s), 3.74-3.50(4 H, m),3.10(1 H, m), 2.92(1 H, dd), 2.52(1 H, dd),1.32(9 H, s), 1.12(3 H, d) | 446 | |
| 29. | 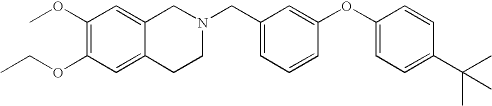 | 2-[3-(4-tert-Butyl-phenoxy)-benzyl]-6-ethoxy-7-methoxy-1,2,3,4-tetrahydroisoquinoline | 7.32(2 H, d), 7.28(1 H, t), 7.10(1 H, d), 7.08(1 H, s),6.96(2 H, d), 6.90(1 H, m), 6.60(1 H, s), 6.50(1 H,s),4.04(2 H, q), 3.80(3 H, s), 3.64(2 H, s), 3.56(2 H, s),2.78(2 H, m), 2.70(2 H, m), 1.44(3 H, t), 1.30(9 H, s) | 446 | * |
| 30. | 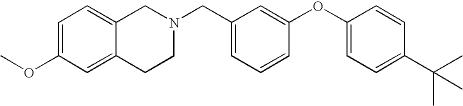 | 2-[3-(4-tert-Butyl-phenoxy)-benzyl]-6-methoxy-1,2,3,4-tetrahydroisoquinoline | 7.32(2 H, d), 7.30(1 H, m), 7.10(1 H, d), 7.08(1 H, s),6.94-6.86(4 H, m), 6.68(1 H, d), 6.62(1 H, s),3.76(3 H, s), 3.64(2 H, s), 3.56(2 H, s), 2.86(2 H, t),2.70(2 H, t), 1.30(9 H, s) | 402 | |
| 31. | 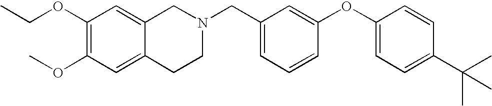 | 2-[3-(4-tert-Butyl-phenoxy)-benzyl]-7-ethoxy-6-methoxy-1,2,3,4-tetrahydroisoquinoline | 7.32(2 H, d), 7.26(1 H, m), 7.12(1 H, d), 7.08(1 H, s),6.94-6.88(3 H, m), 6.60(1 H, s), 6.50(1 H, s),4.02(2 H, q), 3.82(3 H, s), 3.64(2 H, s), 3.54(2 H, s),2.80(2 H, m), 2.70(2 H, m), 1.42(3 H, t), 1.30(9 H, s) | 446 | * |
| 32. | 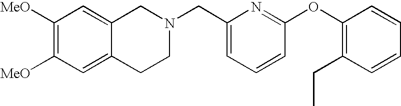 | 2-[6-(2-Ethyl-phenoxy)-pyridin-2-ylmethyl]-6,7-dimethoxy-1,2,3,4-tetrahydroisoquinoline | | 405 | |
| 33. | 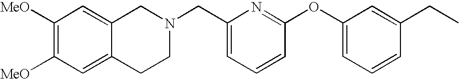 | 2-[6-(3-Ethyl-phenoxy)-pyridin-2-ylmethyl]-6,7-dimethoxy-1,2,3,4-tetrahydroisoquinoline | | 405 | |
| 34. | 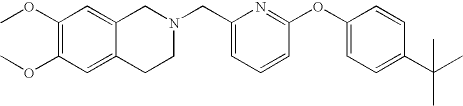 | 2-[6-(4-tert-Butyl-phenoxy)-pyridin-2-ylmethyl]-6,7-dimethoxy-1,2,3,4-tetrahydroisoquinoline | 7.62(1 H, t), 7.38(2 H, d), 7.22(1 H, d), 7.06(2 H, d),6.62(1 H, d), 6.60(1 H, s), 6.50(1 H, s), 3.82(3 H, s),3.80(3 H, s), 3.78(2 H, s), 3.64(2 H, s), 2.82(4 H, m),1.34(9 H, s) | 433 | * |
| 35. | 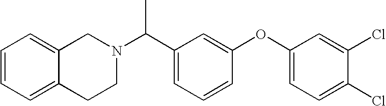 | 2-{1-[3-(3,4-Dichloro-phenoxy)-phenyl]-ethyl}-1,2,3,4-tetrahydroisoquinoline | | 398 | |
| 36. | 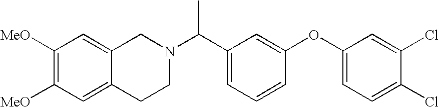 | 2-{1-[3-(3,4-Dichloro-phenoxy)-phenyl]-ethyl}-6,7-dimethoxy-1,2,3,4-tetrahydroisoquinoline | | 458 | |
| 37. | 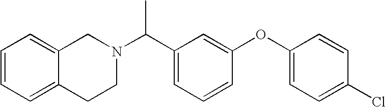 | 2-{1-[3-(4-Chloro-phenoxy)-phenyl]-ethyl}-1,2,3,4-tetrahydroisoquinoline | | 364 | |
| 38. | 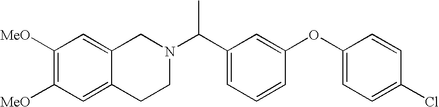 | 2-{1-[3-(4-Chloro-phenoxy)-phenyl]-ethyl}-6,7-dimethoxy-1,2,3,4-tetrahydroisoquinoline | | 424 | |
| 39. | 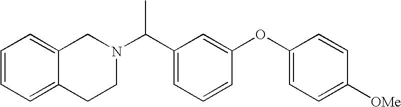 | 2-{1-[3-(4-Methoxy-phenoxy)-phenyl]-ethyl}-1,2,3,4-tetrahydroisoquinoline | | 360 | |
| 40. | 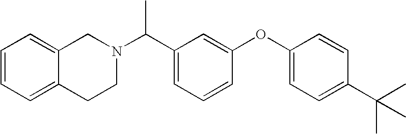 | 2-{1-[3-(4-tert-Butyl-phenoxy)-phenyl]-ethyl}-1,2,3,4-tetrahydroisoquinoline | | 386 | |
| 41. | 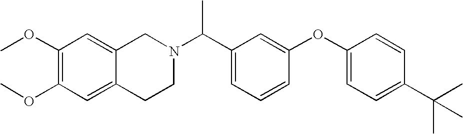 | 2-{1-[3-(4-tert-Butyl-phenoxy)-phenyl]-ethyl}-6,7-dimethoxy-1,2,3,4-tetrahydroisoquinoline | | 446 | |
| 42. | 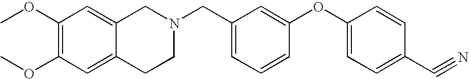 | 4-[3-(6,7-Dimethoxy-3,4-dihydro-1H-isoquinolin-2-ylmethyl)-phenoxy]-benzonitrile | 7.60(2 H, d), 7.36-7.10(3 H, m), 7.06-6.94(3 H, m),6.60(1 H, s), 6.46(1 H, s), 3.84(3 H, s), 3.82(3 H, s),3.68(2 H, s), 3.56(2 H, s), 2.80(2 H, m), 2.74(2 H, m) | 401 | * |
| 43. | 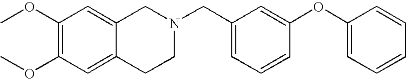 | 6,7-Dimethoxy-2-(3-phenoxy-benzyl)-1,2,3,4-tetrahydroisoquinoline | | 376 | * |
| 44. | 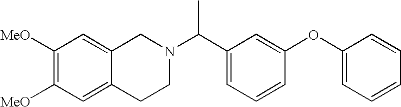 | 6,7-Dimethoxy-2-[1-(3-phenoxy-phenyl)-ethyl]-1,2,3,4-tetrahydroisoquinoline | | 390 | |
| 45. | 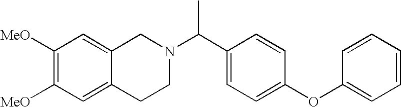 | 6,7-Dimethoxy-2-[1-(4-phenoxy-phenyl)-ethyl]-1,2,3,4-tetrahydroisoquinoline | | 390 | |
| 46. | 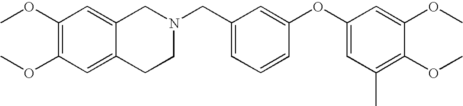 | 6,7-Dimethoxy-2-[3-(3,4,5-trimethoxy-phenoxy)-benzyl]-1,2,3,4-tetrahydroisoquinoline | | 466 | |
| 47. | 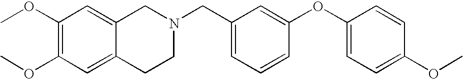 | 6,7-Dimethoxy-2-[3-(4-methoxy-phenoxy)-benzyl]-1,2,3,4-tetrahydroisoquinoline | 7.28(1 H, t), 7.06-6.88(6 H, m), 6.80(1 H, d),6.62(1 H, s), 6.56(1 H, s), 3.72(3 H, s), 3.68(3 H, s),3.66(3 H, s), 3.56(2 H, s), 3.40(2 H, s), 2.70-2.54(4 H, m) | 406 | * |
| 48. | 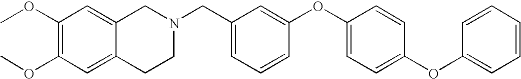 | 6,7-Dimethoxy-2-[3-(4-phenoxy-phenoxy)-benzyl]-1,2,3,4-tetrahydroisoquinoline | | 468 | |
| 49. | 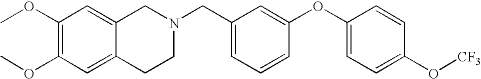 | 6,7-Dimethoxy-2-[3-(4-trifluoromethoxy-phenoxy)-benzyl]-1,2,3,4-tetrahydroisoquinoline | | 460 | * |
| 50. | 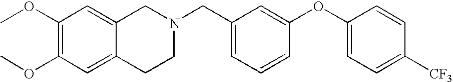 | 6,7-Dimethoxy-2-[3-(4-trifluoromethyl-phenoxy)-benzyl]-1,2,3,4-tetrahydroisoquinoline | | 444 | * |
| 51. | 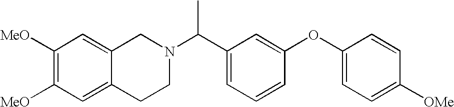 | 6,7-Dimethoxy-2-{1-[3-(4-methoxy-phenoxy)-phenyl]-ethyl}-1,2,3,4-tetrahydroisoquinoline | | 420 | |
| 52. | 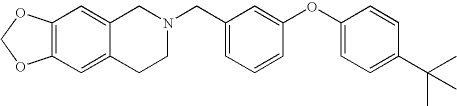 | 6-[3-(4-tert-Butyl-phenoxy)-benzyl]-5,6,7,8-tetrahydro-[1,3]dioxolo[4,5-g]isoquinoline | 7.32(2 H, d), 7.26(1 H, t), 7.10(1 H, d), 7.08(1 H, s),6.96-6.86(3 H, m), 6.56(1 H, s), 6.44(1 H, s),5.86(2 H, s), 3.64(2 H, s), 3.52(2 H, s), 2.78(2 H, m),2.68(2 H, m), 1.30(9 H, s) | 416 | * |
| 53. | 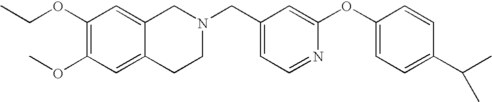 | 7-Ethoxy-2-[2-(4-isopropyl-phenoxy)-pyridin-4-ylmethyl]-6-methoxy-1,2,3,4-tetrahydroisoquinoline | | 433 | * |

### Example 2

### Melanin Concentrating Hormone Receptor Binding Assay

This Example illustrates a standard assay of melanin concentrating hormone receptor binding that may be used to determine the binding affinity of compounds for the MCH receptor.

Cynomolgus macaque hypothalamus MCH1 cDNA is prepared and cloned into PCDNA3.1 (INVITROGEN Corp., Carlsbad, Calif.), and HEK293 cells (American Type Culture Collection, Manassas, Va.) are stably transfected with the MCH1 expression vector as described in PCT International Application publication number WO/03/059289, which published on Jul. 24, 2003. The disclosure of WO 03/059289 at page 52 directed to the preparation and storage of the transfected HEK293 cells is hereby incorporated by reference.

At the time of assay, pellets are thawed by addition of wash buffer (25 mM Hepes with 1.0 mM CaCl₂, 5.0 mM MgCl₂, 120 mM NaCl, pH 7.4) and homogenized for 30 seconds using a BRINKMAN POLYTRON, setting 5. Cells are centrifuged for 10 minutes at 48,000×g. The supernatant is discarded and the pellet is resuspended in fresh wash buffer, and homogenized again. An aliquot of this membrane homogenate is used to determine protein concentration via the Bradford method (BIO-RAD Protein Assay Kit, #500-0001, BIO-RAD, Hercules, Calif.). By this measure, a 1-liter culture of cells typically yields 50-75 mg of total membrane protein. The homogenate is centrifuged as before and resuspended to a protein concentration of 333 μg/mL in binding buffer (Wash buffer+0.1% BSA and 1.0 μM final phosphoramidon) for an assay volume of 50 μg membrane protein/150 μl binding buffer. Phosphoramidon was from SIGMA BIOCHEMICALS, St. Louis, Mo. (cat# R-7385).

Competition binding assays are performed at room temperature in Falcon 96 well round bottom polypropylene plates. Each assay well contains 150 μL of MCH receptor-containing membranes described above, 50 μL ¹²⁵I-Tyr MCH, 50 μL binding buffer, and 2 μL test compound in DMSO. ¹²⁵I-Tyr MCH (specific activity=2200 Ci/mmol) is purchased from NEN (Boston, Mass.) and is diluted in binding buffer to provide a final assay concentration of 30 pM.

Non-specific binding is defined as the binding measured in the presence of 1 μM unlabeled MCH. MCH is purchased from BACHEM U.S.A., King of Prussia, Pa. (cat #H-1482). Assay wells used to determine MCH binding contain 150 μL of MCH receptor containing membranes, 50 μL ¹²⁵I-Tyr MCH, 25 μL binding buffer and 25 μL binding buffer.

Assay plates are incubated for 1 hour at room temperature. Membranes are harvested onto WALLAC™ glass fiber filters (PERKIN-ELMER, Gaithersburg, Md.) which were pre-soaked with 1.0% PEI (polyethyleneimine) for 2 hours prior to use. Filters are allowed to dry overnight, and then counted in a WALLAC 1205 BETA PLATE counter after addition of WALLAC BETA SCINT™ scintillation fluid.

For saturation binding, the concentration of ¹²⁵I-Tyr MCH is varied from 7 to 1,000 pM. Typically, 11 concentration points are collected per saturation binding curve. Equilibrium binding parameters are determined by fitting the allosteric Hill equation to the measured values with the aid of the computer program FitP™ (BIOSOFT, Ferguson, Mo.). For preferred compounds, Kᵢ values are below 1 micromolar, preferably below 500 nanomolar, more preferably below 100 nanomolar.

### Example 3

### Calcium Mobilization Assay

This Example illustrates a representative functional assay for monitoring the response of cells expressing melanin concentrating hormone receptors to melanin concentrating hormone. This assay can also be used to determine if test compounds act as agonists or antagonists of melanin concentrating hormone receptors.

Chinese Hamster Ovary (CHO) cells (American Type Culture Collection; Manassas, Va.) are stably transfected with the MCH expression vector described in Example 2 via calcium phosphate precipitation, and are grown to a density of 15,000 cells/well in FALCON™ black-walled, clear-bottomed 96-well plates (#3904, BECTON-DICKINSON, Franklin Lakes, N.J.) in Ham's F12 culture medium (MEDIATECH, Herndon, Va.) supplemented with 10% fetal bovine serum, 25 mM HEPES and 500 μg/mL (active) G418. Prior to running the assay, the culture medium is emptied from the 96 well plates. Fluo-3 calcium sensitive dye (Molecular Probes, Eugene, Oreg.) is added to each well (dye solution: 1 mg FLUO-3 AM, 440 μL DMSO and 440 μL 20% pluronic acid in DMSO, diluted 1:4, 50 μL diluted solution per well). Plates are covered with aluminum foil and incubated at 37° C. for 1-2 hours. After the incubation, the dye is emptied from the plates, cells are washed once in 100 μL KRH buffer (0.05 mM KCl, 0.115 M NaCl, 9.6 mM NaH₂PO₄, 0.01 mM MgSO₄, 25 mM HEPES, pH 7.4) to remove excess dye; after washing, 80 μL KRH buffer is added to each well.

Fluorescence response is monitored upon the addition of either human MCH receptor or test compound by a FLIPR™ plate reader (Molecular Devices, Sunnyvale, Calif.) by excitation at 480 nm and emission at 530 nm.

In order to measure the ability of a test compound to antagonize the response of cells expressing MCH receptors to MCH, the EC₅₀ of MCH is first determined. An additional 20 μL of KRH buffer and 1 μL DMSO is added to each well of cells, prepared as described above. 100 μL human MCH in KRH buffer is automatically transferred by the FLIPR instrument to each well. An 8-point concentration response curve, with final MCH concentrations of 1 nM to 3 μM, is used to determine MCH EC₅₀.

Test compounds are dissolved in DMSO, diluted in 20 μL KRH buffer, and added to cells prepared as described above. The 96 well plates containing prepared cells and test compounds are incubated in the dark, at room temperature for 0.5-6 hours. It is important that the incubation not continue beyond 6 hours. Just prior to determining the fluorescence response, 100 μL human MCH diluted in KRH buffer to 2×EC₅₀ is automatically added by the FLIPR instrument to each well of the 96 well plate for a final sample volume of 200 μL and a final MCH concentration of EC₅₀. The final concentration of test compounds in the assay wells is between 1 DM and 5 μM. Typically, cells exposed to one EC₅₀ of MCH exhibit a fluorescence response of about 10,000 Relative Fluorescence Units. Cells incubated with antagonists of the MCH receptor exhibit a response that is significantly less than that of the control cells to the p≦0.05 level, as measured using a parametric test of statistical significance. Typically, antagonists of the MCH receptor decrease the fluorescence response by about 20%, preferably by about 50%, and most preferably by at least 80% as compared to matched controls.

The ability of a compound to act as an agonist of the MCH receptor is determined by measuring the fluorescence response of cells expressing MCH receptors, using the methods described above, in the absence of MCH. Compounds that cause cells to exhibit fluorescence above background are MCH receptor agonists.

### Example 4

### MDCK Cytotoxicity Assay

This Example illustrates the evaluation of compound toxicity using a Madin Darby canine kidney (MDCK) cell cytotoxicity assay.

1 μL of test compound is added to each well of a clear bottom 96-well plate (PACKARD, Meriden, Conn.) to give final concentration of compound in the assay of 10 μM, 100 μM or 200 μM. Solvent without test compound is added to control wells.

MDCK cells, ATCC no. CCL-34 (American Type Culture Collection, Manassas, Va.), are maintained in sterile conditions following the instructions in the ATCC production information sheet. Confluent MDCK cells are trypsinized, harvested, and diluted to a concentration of 0.1×10⁶ cells/mL with warm (37° C.) medium (VITACELL Minimum Essential Medium Eagle, ATCC catalog #30-2003). 100 μL of diluted cells is added to each well, except for five standard curve control wells that contain 100 μL of warm medium without cells. The plate is then incubated at 37° C. under 95% O₂, 5% CO₂ for 2 hours with constant shaking. After incubation, 50 μL of mammalian cell lysis solution (from the PACKARD (Meriden, Conn.) ATP-LITE-M Luminescent ATP detection kit) is added per well, the wells are covered with PACKARD TOPSEAL stickers, and plates are shaken at approximately 700 rpm on a suitable shaker for 2 minutes.

Compounds causing toxicity will decrease ATP production, relative to untreated cells. The ATP-LITE-M Luminescent ATP detection kit is generally used according to the manufacturer's instructions to measure ATP production in treated and untreated MDCK cells. PACKARD ATP LITE-M reagents are allowed to equilibrate to room temperature. Once equilibrated, the lyophilized substrate solution is reconstituted in 5.5 mL of substrate buffer solution (from kit). Lyophilized ATP standard solution is reconstituted in deionized water to give a 10 mM stock. For the five control wells, 10 μL of serially diluted PACKARD standard is added to each of the standard curve control wells to yield a final concentration in each subsequent well of 200 nM, 100 nM, 50 nM, 25 nM, and 12.5 nM. PACKARD substrate solution (50 μL) is added to all wells, which are then covered, and the plates are shaken at approximately 700 rpm on a suitable shaker for 2 minutes. A white PACKARD sticker is attached to the bottom of each plate and samples are dark adapted by wrapping plates in foil and placing in the dark for 10 minutes. Luminescence is then measured at 22° C. using a luminescence counter (e.g., PACKARD TOPCOUNT Microplate Scintillation and Luminescence Counter or TECAN SPECTRAFLUOR PLUS), and ATP levels calculated from the standard curve. ATP levels in cells treated with test compound(s) are compared to the levels determined for untreated cells. Cells treated with 10 μM of a preferred test compound exhibit ATP levels that are at least 80%, preferably at least 90%, of the ATP levels detected in untreated cells. When a 100 μM concentration of the test compound is used, cells treated with preferred test compounds exhibit ATP levels that are at least 50%, preferably at least 80%, of the ATP levels detected in untreated cells.

### Example 5

### Microsomal In Vitro Half-Life

This Example illustrates the evaluation of compound half-life values (t_{1/2} values) using a representative liver microsomal half-life assay.

Pooled human liver microsomes are obtained from XenoTech LLC (Kansas City, Kans.). Such liver microsomes may also be obtained from In Vitro Technologies (Baltimore, Md.) or Tissue Transformation Technologies (Edison, N.J.). Six test reactions are prepared, each containing 25 μL microsomes, 5 μL of a 100 μM solution of test compound, and 399 μL 0.1 M phosphate buffer (19 mL 0.1 M NaH₂PO₄, 81 mL 0.1 M Na₂HPO₄, adjusted to pH 7.4 with H₃PO₄). A seventh reaction is prepared as a positive control containing 25 μL microsomes, 399 μL 0.1 M phosphate buffer, and 5 mL of a 100 μM solution of a compound with known metabolic properties (e.g., DIAZEPAM or CLOZAPINE). Reactions are preincubated at 39° C. for 10 minutes.

Cofactor mixture is prepared by diluting 16.2 mg NADP and 45.4 mg glucose-6-phosphate in 4 mL 100 mM MgCl₂. Glucose-6-phosphate dehydrogenase solution is prepared by diluting 214.3 μL glucose-6-phosphate dehydrogenase suspension (Roche Molecular Biochemicals; Indianapolis, Ind.) into 1285.7 μL distilled water. 71 μL of starting reaction mixture (3 mL cofactor mixture; 1.2 mL glucose-6-phosphate dehydrogenase solution) is added to 5 of the 6 test reactions and to the positive control. 71 μL 100 mM MgCl₂ is added to the sixth test reaction, which is used as a negative control. At each time point (0, 1, 3, 5, and 10 minutes), 75 μL of each reaction mix is pipetted into a well of a 96-well deep-well plate containing 75 μL ice-cold acetonitrile. Samples are vortexed and centrifuged 10 minutes at 3500 rpm (Sorval T 6000D centrifuge, H1000B rotor). 75 μL of supernatant from each reaction is transferred to a well of a 96-well plate containing 150 μL of a 0.5 μM solution of a compound with a known LCMS profile (internal standard) per well. LCMS analysis of each sample is carried out and the amount of unmetabolized test compound is measured as AUC, compound concentration vs. time is plotted, and the t_{1/2} value of the test compound is extrapolated. Preferred compounds provided herein exhibit in vitro t_{1/2} values of greater than 10 minutes and less than 4 hours, preferably between 30 minutes and 1 hour, in human liver microsomes.

1. A compound of the formula 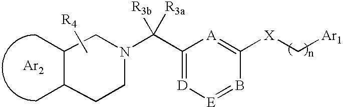
or a pharmaceutically acceptable salt thereof, wherein:
A, B, E, and D are independently CR₂ or N;
n is 0 or 1;
X is O, NH or CH₂;
Ar₁ is phenyl or a 6-membered aromatic heterocycle, each of which is substituted with from 0 to 4 substituents independently chosen from Rₐ; or two adjacent substituents are taken together to form, with the ring atoms to which they are bound, a fused 5- or 6-membered ring substituted with from 0 to 4 substituents independently chosen from Rₐ; 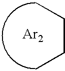
represents fused phenyl or a fused 6-membered aromatic heterocycle, each of which is substituted with from 0 to 4 substituents independently chosen from Rₐ; or two adjacent substituents are taken together to form, with the ring atoms to which they are bound, a fused 5- or 6-membered ring substituted with from 0 to 3 substituents independently chosen from Rₐ;
R₂ is independently chosen at each occurrence from hydrogen, hydroxy, halogen, amino, nitro, cyano, C₁-C₆alkyl, C₂-C₆alkenyl, C₂-C₆alkynyl, C₁-C₆alkoxy, C₃-C₇cycloalkyl(C₀-C₄alkyl), C₁-C₆haloalkyl, C₁-C₆haloalkoxy, and mono- and di-(C₁-C₄alkyl)amino;
R₃ₐ and R_{3b} are independently hydrogen, hydroxy, halogen, cyano, C₁-C₆alkyl, C₁-C₆alkenyl, C₂-C₆alkynyl, C₁-C₆alkoxy, C₃-C₇cycloalkyl(C₀-C₄alkyl), C₁-C₆haloalkyl or C₁-C₆haloalkoxy; or R₃ₐ and R_{3b} are taken together to form an oxo group;
R₄ represents from 0 to 3 substituents independently chosen from hydroxy, halogen, amino, nitro, cyano, C₁-C₆alkyl, C₂-C₆alkenyl, C₂-C₆alkynyl, C₁-C₆alkoxy, C₃-C₇cycloalkyl(C₀-C₄alkyl), C₁-C₆haloalkyl, and C₁-C₆haloalkoxy; and
Rₐ is independently chosen at each occurrence from:
(i) hydroxy, halogen, amino, aminocarbonyl, cyano, nitro, and —COOH; and
(ii) C₁-C₈alkyl, C₂-C₈alkenyl, C₂-C₈alkynyl, C₁-C₈alkoxy, C₃-C₇cycloalkyl(C₀-C₄alkyl), C₁-C₈haloalkyl, C₁-C₈haloalkoxy, C₁-C₈alkanoyl, C₃-C₈alkanone, C₂-C₈alkoxycarbonyl, C₂-C₈alkanoyloxy, C₁-C₈alkylthio, C₂-C₈alkyl ether, phenylC₁-C₄alkyl, phenylC₀-C₄alkoxy, mono- and di-(C₁-C₆alkyl)aminoC₀-C₆alkyl, and (4- to 7-membered heterocycle)C₁-C₄alkyl; each of which is substituted with from 0 to 3 substituents independently chosen from hydroxy, halogen, amino, cyano, C₁-C₄alkyl, C₁-C₄alkoxy, C₁-C₄haloalkyl, C₁-C₄haloalkoxy, and mono- and di-(C₁-C₄alkyl)amino;
and wherein if:
(i) 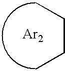
is unsubstituted phenyl, di-methoxy substituted phenyl, or phenyl substituted with phenyl (C₁-C₂alkoxy); and
(ii) A, B, E, and D are each CR₂; 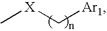
and Ar₁ is phenyl,
then Ar₁ is substituted at the position para to the point of attachment with a substituent other than halogen.

2. A compound or salt according to claim 1, wherein Ar₁ is phenyl or pyridyl, each of which is substituted with from 0 to 4 substituents independently chosen from hydroxy, halogen, cyano, nitro, amino, C₁-C₆alkyl, C₃-C₇cycloalkyl(C₀-C₄alkyl), C₁-C₆haloalkyl, C₁-C₆alkoxy, C₁-C₆haloalkoxy, C₁-C₆alkylthio, C₂-C₆alkyl ether, mono- and di-(C₁-C₆alkyl)amino, phenyl and phenoxy.

3. A compound or salt according to claim 2, wherein Ar₁ is substituted with 1, 2 or 3 substituents independently chosen from hydroxy, halogen, cyano, nitro, amino, C₁-C₄alkyl, C₁-C₄alkoxy, C₃-C₇cycloalkyl(C₀-C₂alkyl), C₁-C₄haloalkyl, C₀-C₄haloalkoxy and phenoxy.

4. A compound or salt according to claim 1, wherein 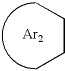
represents a fused ring chosen from phenyl and pyridyl, each of which is substituted with from 0 to 4 substituents independently chosen from hydroxy, halogen, cyano, nitro, amino, C₁-C₆alkyl, C₁-C₆alkoxy, C₃-C₇cycloalkyl(C₀-C₄alkyl), C₁-C₆haloalkyl, C₁-C₆haloalkoxy, C₁-C₆alkylthio, C₂-C₆alkyl ether, and mono- and di-(C₁-C₆alkyl)amino.

5-6. (canceled)

7. A compound or salt according to claim 6, wherein Ar₂ is phenyl or pyridyl, each of which is substituted with from 0 to 4 substituents independently chosen from hydroxy, halogen, cyano, nitro, amino, C₁-C₆alkyl, C₁-C₆alkoxy, C₃-C₇cycloalkyl(C₁-C₄alkyl), C₁-C₆haloalkyl, C₁-C₆haloalkoxy, C₁-C₆alkylthio, C₂-C₆alkyl ether, mono- and di-(C₁-C₆alkyl)amino, phenyl and phenoxy.

8. A compound or salt according to claim 1, wherein each R₂ is independently chosen from hydrogen, halogen, amino, hydroxy, cyano, nitro, C₁-C₄alkyl, C₁-C₄alkoxy, C₃-C₇cycloalkyl(C₀-C₂alkyl), C₁-C₄haloalkyl and C₁-C₄haloalkoxy.

9. A compound or salt according to claim 8, wherein A, B, E, and D are each CR₂.

10. A compound or salt according to claim 8, wherein 1 or 2 of A, B, F, and D is N, and the remainder are CR₂.

11. A compound or salt according to claim 1, wherein R₃ₐ is hydrogen, C₁-C₄alkyl, C₂-C₄alkenyl, C₂-C₄alkynyl, C₃-C₇cycloalkyl(C₀-C₂alkyl) or C₁-C₄haloalkyl; and R_{3b} is hydrogen.

12. (canceled)

13. A compound according to claim 1, wherein R₃ₐ and R_{3b} are taken together to form an oxo group.

14. A compound or salt according to claim 1, wherein R₄ is 0, 1 or 2 substituents independently chosen from hydroxy, halogen, cyano, C₁-C₄alkyl, C₂-C₄alkenyl, C₁-C₄alkynyl, C₁-C₄alkoxy, C₃-C₇cycloalkyl(C₀-C₂alkyl), C₁-C₄haloalkyl and C₁-C₄haloalkoxy.

15. (canceled)

16. A compound or salt according to claim 1, wherein n is 0.

17. A compound or salt according to claim 1, wherein n is 1.

18. A compound or salt according to claim 8, wherein:
Ar₁ is phenyl or pyridyl, each of which is substituted with from 0 to 4 substituents independently chosen from hydroxy, halogen, cyano, nitro, amino, C₁-C₆alkyl, C₁-C₆haloalkyl, C₁-C₆alkoxy, C₃-C₇cycloalkyl(C₀-C₄alkyl), C₁-C₆haloalkoxy, C₁-C₆alkylthio, C₁-C₆alkyl ether, mono- and di-(C₁-C₆alkyl)amino, phenyl and phenoxy; 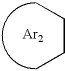
represents phenyl or pyridyl, each of which is substituted with from 0 to 4 substituents independently chosen from hydroxy, halogen, cyano, nitro, amino, C₁-C₆alkyl, C₁-C₆haloalkyl, C₁-C₆alkoxy, C₃-C₇cycloalkyl(C₀-C₄alkyl), C₁-C₆haloalkoxy, C₁-C₆alkylthio, C₂-C₆alkyl ether, and mono- and di-(C₁-C₆alkyl)amino; and
R₄ represents 0, 1 or 2 substituents independently chosen from hydroxy, halogen, cyano, C₁-C₄alkyl, C₂-C₄alkenyl, C₁-C₄alkynyl, C₁-C₄alkoxy, C₃-C₇cycloalkyl(C₀-C₂alkyl), C₁-C₄haloalkyl, and C₁-C₄haloalkoxy.

19. A compound or salt according to claim 1, having the formula: 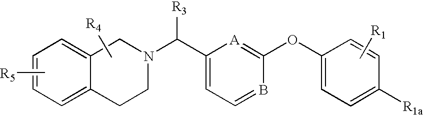
wherein:
A and B are independently CR₂ or N;
each R₂ is independently chosen from hydrogen, halogen, amino, hydroxy, cyano, nitro, C₁-C₄alkyl, C₁-C₄alkoxy, C₃-C₇cycloalkyl(C₀-C₂alkyl), C₁-C₄haloalkyl and C₁-C₄haloalkoxy;
R₁ₐ is hydrogen, hydroxy, halogen, cyano, nitro, amino, C₁-C₆alkyl, C₁-C₆haloalkyl, C₁-C₆alkoxy, C₃-C₇cycloalkyl(C₀-C₄alkyl), C₁-C₆haloalkoxy, C₁-C₆alkylthio, C₂-C₆alkyl ether, mono- or di-(C₁-C₆alkyl)amino, phenyl or phenoxy;
R₁ represents from 0 to 3 substituents independently chosen from hydroxy, halogen, cyano, nitro, amino, C₁-C₆alkyl, C₁-C₆haloalkyl, C₁-C₆alkoxy, C₃-C₇cycloalkyl(C₀-C₄alkyl), C₁-C₆haloalkoxy, C₁-C₆alkylthio, C₂-C₆alkyl ether, mono- and di-(C₁-C₆alkyl)amino, phenyl, and phenoxy;
R₃ is hydrogen, C₁-C₆alkyl, C₂-C₆alkenyl, C₂-C₆alkynyl or C₁-C₆haloalkyl;
R₄ represents 0, 1 or 2 substituents independently chosen from hydroxy, halogen, cyano, C₁-C₄alkyl, C₂-C₄alkenyl, C₁-C₄alkynyl, C₁-C₄alkoxy, C₃-C₇cycloalkyl(C₀-C₂alkyl), C₁-C₄haloalkyl, and C₁-C₄haloalkoxy; and
R₅ represents from 0 to 4 substituents independently chosen from Rₐ; or two adjacent R₅ are taken together to form a fused 5- or 6-membered ring substituted with from 0 to 4 substituents independently chosen from Rₐ.

20. A compound or salt according to claim 19, wherein one of A and B is nitrogen.

21. A compound or salt according, to claim 19, wherein A and B are CH.

22. A compound or salt according to claim 19, wherein R₁ₐ is hydroxy, halogen, cyano, C₁-C₆alkyl, C₁-C₆alkoxy, C₃-C₇cycloalkyl(C₀-C₄alkyl), C₁-C₆haloalkyl, C₁-C₆haloalkoxy or phenoxy.

23. (canceled)

24. A compound or salt according to claim 19, wherein R₅ represents from 1 to 3 substituents independently chosen from hydroxy, halogen, cyano, nitro, amino, C₁-C₆alkyl, C₁-C₆haloalkyl, C₁-C₆alkoxy, C₃-C₇cycloalkyl(C₀-C₄alkyl), C₁-C₆haloalkoxy, C₁-C₆alkylthio, C₂-C₆alkyl ether, and mono- and di-(C₁-C₆alkyl)amino.

25. A compound or salt according to claim 1, having the formula: 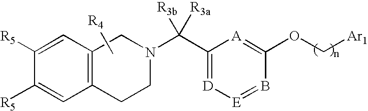
wherein each R₅ is independently chosen from hydrogen, hydroxy, halogen, cyano, nitro, amino, C₁-C₆alkyl, C₁-C₆haloalkyl, C₁-C₆alkoxy, C₃-C₇cycloalkyl(C₀-C₄alkyl), C₁-C₆haloalkoxy, C₁-C₆alkylthio, C₂-C₆alkyl ether, and mono- and di-(C₁-C₆alkyl)amino.

26. A compound or salt according to claim 1, having the formula: 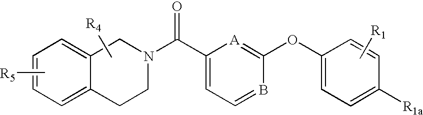
wherein:
A and B are independently CR₂ or N;
each R₂ is independently chosen from hydrogen, halogen, amino, hydroxy, cyano, nitro, C₁-C₄alkyl, C₁-C₄alkoxy, C₃-C₇cycloalkyl(C₀-C₂alkyl), C₁-C₄haloalkyl and C₁-C₄haloalkoxy.
R₁ₐ is hydroxy, halogen, cyano, nitro, amino, C₁-C₆alkyl, C₁-C₆haloalkyl, C₁-C₆alkoxy, C₃-C₇cycloalkyl(C₀-C₄alkyl), C₁-C₆haloalkoxy, C₁-C₆alkylthio, C₂-C₆alkyl ether, mono- or di-(C₁-C₆alkyl)amino, phenyl and phenoxy;
R₁ represents from 0 to 3 substituents independently chosen from hydroxy, halogen, cyano, nitro, amino, C₁-C₆alkyl, C₁-C₆haloalkyl, C₁-C₆alkoxy, C₃-C₇cycloalkyl(C₀-C₄alkyl), C₁-C₆haloalkoxy, C₁-C₆alkylthio, C₂-C₆alkyl ether, mono- and di-(C₁-C₆alkyl)amino, phenyl and phenoxy;
R₄ represents 0, 1 or 2 substituents independently chosen from hydroxy, halogen, cyano, C₁-C₄alkyl, C₂-C₄alkenyl, C₂-C₄alkynyl, C₁-C₄alkoxy, C₃-C₇cycloalkyl(C₁-C₂alkyl), C₁-C₄haloalkyl and C₁-C₄haloalkoxy; and
R₅ represents from 0 to 4 substituents independently chosen from Rₐ; or two adjacent R₅ are taken together to form a fused 5- or 6-membered ring substituted with from 0 to 4 substituents independently chosen from Rₐ.

27. A compound or salt according to claim 1, having the formula: 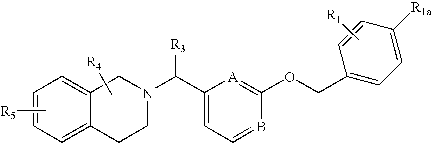
wherein;
A and B are independently CR₂ or N;
each R₂ is independently chosen from hydrogen, halogen, amino, hydroxy, cyano, nitro, C₁-C₄alkyl, C₁-C₄alkoxy, C₃-C₇cycloalkyl(C₀-C₂alkyl), C₁-C₄haloalkyl, and C₁-C₄haloalkoxy;
R₁ₐ is hydrogen, hydroxy, halogen, cyano, nitro, amino, C₁-C₆alkyl, C₁-C₆haloalkyl, C₁-C₆alkoxy, C₃-C₇cycloalkyl(C₀-C₄alkyl), C₁-C₆haloalkoxy, C₁-C₆alkylthio, C₂-C₆alkyl ether, mono- and di-(C₁-C₆alkyl)amino, phenyl or phenoxy;
R₁ represents from 0 to 3 substituents independently chosen from hydroxy, halogen, cyano, nitro, amino, C₁-C₆alkyl, C₁-C₆haloalkyl, C₁-C₆alkoxy, C₃-C₇cycloalkyl(C₀-C₄alkyl), C₁-C₆haloalkoxy, C₁-C₆alkylthio, C₂-C₆alkyl ether, mono- and di-(C₁-C₆alkyl)amino, phenyl, and phenoxy;
R₃ is hydrogen, C₁-C₆alkyl, C₁-C₆alkenyl, C₂-C₆alkynyl or C₁-C₆haloalkyl;
R₄ represents 0, 1 or 2 substituents independently chosen from hydroxy, halogen, cyano, C₁-C₄alkyl, C₂-C₄alkenyl, C₂-C₄alkynyl, C₁-C₄alkoxy, C₃-C₇cycloalkyl(C₀-C₄alkyl), C₁-C₄haloalkyl and C₁-C₄haloalkoxy; and
R₅ represents from 0 to 4 substituents independently chosen from Rₐ; or two adjacent R₅ are taken together to form a fused 5- or 6-membered ring substituted with from 0 to 4 substituents independently chosen from Rₐ.

28. A compound or salt according to claim 1, having the formula: 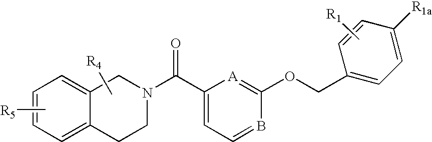
wherein:
A and B are independently CR₂ or N;
each R₂ is independently chosen from hydrogen, halogen, amino, hydroxy, cyano, nitro, C₁-C₄alkyl, C₁-C₄alkoxy, C₁-C₄haloalkyl, and C₁-C₄haloalkoxy;
R₁ₐ is hydrogen, hydroxy, halogen, cyano, nitro, amino, C₁-C₆alkyl, C₁-C₆haloalkyl, C₁-C₆alkoxy, C₁-C₆haloalkoxy, C₁-C₆alkylthio, C₃-C₇cycloalkyl(C₀-C₂alkyl), C₂-C₆alkyl ether, mono- or di-(C₁-C₆alkyl)amino, phenyl or phenoxy;
R₁ represents from 0 to 3 substituents independently chosen from hydroxy, halogen, cyano, nitro, amino, C₁-C₆alkyl, C₁-C₆haloalkyl, C₁-C₆alkoxy, C₃-C₇cycloalkyl(C₀-C₄alkyl), C₁-C₆haloalkoxy, C₁-C₆alkylthio, C₂-C₆alkyl ether, mono- and di-(C₁-C₆alkyl)amino phenyl and phenoxy;
R₄ represents 0, 1 or 2 substituents independently chosen from hydroxy, halogen, cyano, C₁-C₄alkyl, C₂-C₄alkenyl, C₂-C₄alkynyl, C₁-C₄alkoxy, C₃-C₇cycloalkyl(C₀-C₂alkyl), C₁-C₄haloalkyl and C₁-C₄haloalkoxy; and
R₅ represents from 0 to 4 substituents independently chosen from Rₐ; or two adjacent R₅ are taken together to form a fused 5- or 6-membered ring substituted with from 0 to 4 substituents independently chosen from Rₐ.

29. A compound or salt according to claim 1, having the formula: 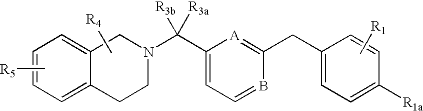
wherein:
A and B are independently CR₂ or N;
each R₂ is independently chosen from hydrogen, halogen, amino, hydroxy, cyano, nitro, C₁-C₄alkyl, C₁-C₄alkoxy, C₃-C₇cycloalkyl(C₀-C₂alkyl), C₁-C₄haloalkyl, and C₁-C₄haloalkoxy;
R₁ₐ is hydrogen, hydroxy, halogen, cyano, nitro, amino, C₁-C₆alkyl, C₁-C₆haloalkyl, C₁-C₆alkoxy, C₃-C₇cycloalkyl(C₀-C₄alkyl), C₁-C₆haloalkoxy, C₁-C₆alkylthio, C₂-C₆alkyl ether, mono- or di-(C₁-C₆alkyl)amino, phenyl or phenoxy;
R₁ represents from 0 to 3 substituents independently chosen from hydroxy, halogen, cyano, nitro, amino, C₁-C₆alkyl, C₁-C₆haloalkyl, C₁-C₆alkoxy, C₃-C₇cycloalkyl(C₀-C₄alkyl), C₁-C₆haloalkoxy, C₁-C₆alkylthio, C₁-C₆alkyl ether, mono- and di-(C₁-C₆alkyl)amino, phenyl and phenoxy;
R₃ₐ and R_{3b} are independently hydrogen, hydroxy, halogen, cyano, C₁-C₆alkyl, C₂-C₆alkenyl, C₂-C₆alkynyl or C₁-C₆haloalkyl; or R₃ₐ and R_{3b} are taken together to form an oxo group;
R₄ represents 0.1 or 2 substituents independently chosen from hydroxy, halogen, cyano, C₁-C₄alkyl, C₂-C₄alkenyl, C₁-C₄alkynyl, C₁-C₄alkoxy, C₃-C₇cycloalkyl(C₀-C₂alkyl), C₁-C₄haloalkyl and C₁-C₄haloalkoxy; and
R₅ represents from 0 to 4 substituents independently chosen from Rₐ; or two adjacent R₅ are taken together to form a fused 5- or 6-membered ring substituted with from 0 to 4 substituents independently chosen from Rₐ.

30. A compound or pharmaceutically acceptable salt thereof having the formula: 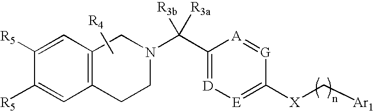
wherein
A, C, E, and D are independently CR₂ or N;
n is 0 or 1;
X is oxygen or CH₂;
Ar₁ is phenyl or a 6-membered aromatic heterocycle, each of which is substituted with from 0 to 4 substituents independently chosen from Rₐ; or two adjacent substituents are taken together to form, with the ring atoms to which they are bound, a fused 5- or 6-membered ring substituted with from 0 to 4 substituents independently chosen from Rₐ;
R₂ is independently chosen at each occurrence from hydrogen, halogen, amino, hydroxy, cyano, nitro, C₁-C₄alkyl, C₁-C₄alkoxy, C₃-C₇cycloalkyl(C₀-C₂alkyl), C₁-C₄haloalkyl and C₁-C₄haloalkoxy;
R₃ₐ is hydroxy, halogen, cyano, C₁-C₆alkyl, C₂-C₆alkenyl, C₂-C₆alkynyl, C₁-C₆alkoxy, C₃-C₇cycloalkyl(C₀-C₄alkyl), C₁-C₆haloalkyl or C₁-C₆haloalkoxy;
R_{3b} is hydrogen, hydroxy, halogen, cyano, C₁-C₆alkyl, C₂-C₆alkenyl, C₂-C₆alkynyl, C₁-C₆alkoxy, C₃-C₇cycloalkyl(C₀-C₄alkyl), C₁-C₆haloalkyl or C₁-C₆haloalkoxy; or R₃ₐ and R_{3b} are taken together to form an oxo group;
R₄ represents from 0 to 3 substituents independently chosen from hydroxy, halogen, amino, nitro, cyano, C₁-C₆alkyl, C₂-C₆alkenyl, C₂-C₆alkynyl, C₁-C₆alkoxy, C₃-C₇cycloalkyl(C₀-C₄alkyl), C₁-C₆haloalkyl and C₁-C₆haloalkoxy;
R₅ is independently chosen from hydroxy, halogen, cyano, nitro, amino, C₁-C₆alkyl, C₁-C₆haloalkyl, C₁-C₆alkoxy, C₃-C₇cycloalkyl(C₀-C₄alkyl), C₁-C₆haloalkoxy, C₁-C₆alkylthio, C₂-C₆alkyl ether, and mono- and di-(C₁-C₆alkyl)amino, and
Rₐ is independently chosen at each occurrence from:
(i) hydroxy, halogen, amino, aminocarbonyl, cyano, nitro, and COOH; and
(ii) C₁-C₈alkyl, C₂-C₈alkenyl, C₂-C₈alkynyl, C₁-C₈alkoxy, C₃-C₇cycloalkyl(C₁-C₄alkyl), C₁-C₈haloalkyl, C₁-C₈haloalkoxy, C₁-C₈alkanoyl, C₃-C₈alkanone, C₂-C₈alkoxycarbonyl, C₂-C₈alkanoyloxy, C₁-C₈alkylthio, C₂-C₈alkyl ether, phenylC₀-C₄alkyl, phenylC₀-C₄alkoxy, mono- and di-(C₁-C₆alkyl)aminoC₀-C₆alkyl, and (4- to 7-membered heterocycle)C₀-C₄alkyl; each of which is substituted with from 0 to 3 substituents independently chosen from hydroxy, halogen, amino, cyano, C₁-C₄alkyl, C₁-C₄alkoxy, C₁-C₄haloalkyl, C₁-C₄haloalkoxy, and mono- and di-(C₁-C₄alkyl)amino.

31. A compound or salt according to claim 30, having the formula: 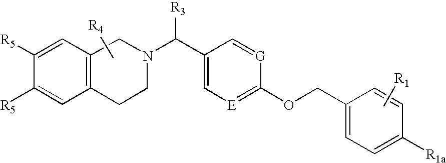
wherein
G and E are independently CR₂ or N;
each R₂ is independently chosen from hydrogen, halogen, amino, hydroxy, cyano, nitro, C₁-C₄alkyl, C₁-C₄alkoxy, C₃-C₇cycloalkyl(C₀-C₂alkyl), C₁-C₄haloalkyl, and C₁-C₄haloalkoxy;
R₁ₐ is hydrogen, hydroxy, halogen, cyano, nitro, amino, C₁-C₆alkyl, C₁-C₆haloalkyl, C₁-C₆alkoxy, C₃-C₇cycloalkyl(C₀-C₄alkyl), C₁-C₆haloalkoxy, C₁-C₆alkylthio, C₂-C₆alkyl ether, mono- or di-(C₁-C₆alkyl)amino, phenyl or phenoxy;
R₁ represents from 0 to 3 substituents independently chosen from hydroxy, halogen, cyano, nitro, amino, C₁-C₆alkyl, C₁-C₆haloalkyl, C₁-C₆alkoxy, C₃-C₇cycloalkyl(C₀-C₄alkyl), C₁-C₆haloalkoxy, C₁-C₆alkylthio, C₂-C₆alkyl ether, mono- and di-(C₁-C₆alkyl)amino, phenyl, and phenoxy;
R₃ is C₁-C₆alkyl, C₂-C₆alkenyl, C₂-C₆alkynyl or C₁-C₆haloalkyl;
R₄ represents 0, 1 or 2 substituents independently chosen from hydroxy, halogen, cyano, C₁-C₄alkyl, C₂-C₄alkenyl, C₂-C₄alkynyl, C₁-C₄alkoxy, C₃-C₇cycloalkyl(C₀-C₂alkyl), C₁-C₄haloalkyl and C₁-C₄haloalkoxy; and
R₅ is independently chosen at each occurrence from Rₐ.

32. A compound or salt according to claim 30 wherein X is oxygen and A, G, D, and E are all CR₂.

33. A compound or salt according to claim 31, wherein at least one of G and E is nitrogen.

34. A compound or salt according to claim 1, wherein the compound exhibits a Kᵢ of 1 micromolar or less in a MCH receptor ligand binding assay.

35-37. (canceled)

38. A pharmaceutical composition, comprising a compound or salt according to claim 1, in combination with at least one physiologically acceptable carrier or excipient.

39. A pharmaceutical composition according to claim 38, wherein the composition is formulated as an injectable fluid, an aerosol, a cream, a gel, a pill, a capsule, a syrup or a transdermal patch.

40. A packaged pharmaceutical preparation, comprising:
(a) a pharmaceutical composition comprising at least one physiologically acceptable carrier or excipient together with a compound of the formula: 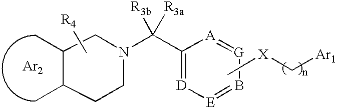
or a pharmaceutically acceptable salt thereof; wherein:
A, E, and D are independently CR₂ or N; and one of B and G is chosen from CR₂ and N; and the
other of B and G is a carbon atom covalently bound to the group 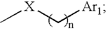
X is O, NH or CH₂;
n is 0 or 1;
Ar₁ is phenyl or a 6-membered aromatic heterocycle, each of which is substituted with from 0 to 4 substituents independently chosen from Rₐ; or two adjacent substitutents are taken together to form, with the ring atoms to which they are bound, a fused 5- or 6-membered ring substituted with from 0 to 4 substituents independently chosen from Rₐ; 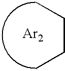
Represents fused phenyl or a fused 6-membered aromatic heterocycle, each of which is substituted with from 0 to 4 substituents independently chosen from Rₐ; or two adjacent substituents are taken together to form, with the ring atoms to which they are bound, a fused 5- or 6-membered ring substituted with from 0 to 3 substituents independently chosen from Rₐ;
R₂ is independently chosen at each occurrence from hydrogen, hydroxy, halogen, amino, nitro, cyano, C₁-C₆alkyl, C₂-C₆alkenyl, C₂-C₆alkynyl, C₁-C₆alkoxy, C₃-C₇cycloalkyl(C₀-C₄alkyl), C₁-C₆haloalkyl, C₁-C₆haloalkoxy, and mono- and di-(C₁-C₄alkyl)amino;
R₃ₐ and R_{3b} are independently hydrogen, hydroxy, halogen, cyano, C₁-C₆alkyl, C₂-C₆alkenyl, C₂-C₆alkynyl, C₁-C₆alkoxy, C₃-C₇cycloalkyl(C₀-C₄alkyl), C₁-C₆haloalkyl or C₁-C₆haloalkoxy; or R₃ₐ and R_{3b} are taken together to form an oxo group;
R₄ represents from 0 to 3 substituents independently chosen from hydroxy, halogen, amino, nitro, cyano, C₁-C₆alkyl, C₂-C₆alkenyl, C₂-C₆alkynyl, C₁-C₆alkoxy, C₃-C₇cycloalkyl(C₀-C₄alkyl), C₁-C₆haloalkyl, and C₁-C₆haloalkoxy;
Rₐ is independently chosen at each occurrence from:
(i) hydroxy, halogen, amino, aminocarbonyl, cyano, nitro, and —COOH; and
(ii) C₁-C₈alkyl, C₂-C₈alkenyl, C₂-C₈alkynyl, C₁-C₈alkoxy, C₃-C₇cycloalkyl(C₀-C₄alkyl), C₁-C₈haloalkyl, C₁-C₈haloalkoxy, C₁-C₈alkanoyl, C₃-C₈alkanone, C₂-C₈alkoxycarbonyl, C₂-C₈alkanoyloxy, C₁-C₈alkylthio, C₂-C₈alkyl ether, phenylC₀-C₄alkyl, phenylC₀-C₄alkoxy, mono- and di-(C₁-C₆alkyl)aminoC₀-C₆alkyl, and (4- to 7-membered heterocycle)C₀-C₄alkyl; each of which is substituted with from 0 to 3 substituents independently chosen from hydroxy, halogen, amino, cyano, C₁-C₄alkyl, C₁-C₄alkoxy, C₁-C₄haloalkyl, C₁-C₄haloalkoxy, and mono- and di-(C₁-C₄alkyl)amino;
(b) in a container; and
(c) instructions for using the composition to treat a patient suffering from a disorder associated with MCH receptor activation.

41. A packaged pharmaceutical preparation according to claim 40, wherein the disorder is an eating disorder, sexual disorder, obesity, diabetes, heart disease or stroke.

42-52. (canceled)

53. A method for treating a disease or disorder associated with MCH receptor activation, comprising administering to a patient in need of such treatment a therapeutically, effective amount of a compound of the formula 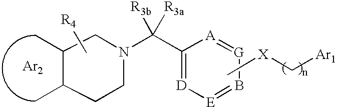
or a pharmaceutically acceptable salt thereof; wherein:
A, E, and D are independently CR₂ or N; and one of B and G is chosen from CR₂ and N; and the
other of B and G is a Carbon atom covalently bound to the group 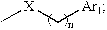
X is O, NH or CH₂;
n is 0 or 1;
Ar₁ is phenyl or a 6-membered aromatic heterocycle, each of which is substituted with from 0 to 4 substituents independently chosen from Rₐ; or two adjacent substituents are taken together to form, with the ring atoms to which they are bound, a fused 5- or 6-membered ring substituted with from 0 to 4 substituents independently chosen from Rₐ; 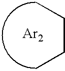
represents fused phenyl or a fused 6-membered aromatic heterocycle, each of which is substituted with from 0 to 4 substituents independently chosen from Rₐ; or two adjacent substituents are taken together to form, with the ring atoms to which they are bound, a fused 5- or 6-membered ring substituted with from 0 to 3 substituents independently chosen from Rₐ;
R₂ is independently chosen at each occurrence from hydrogen, hydro-oxy, halogen, amino, nitro, cyano, C₁-C₆alkyl, C₂-C₆alkenyl, C₂-C₆alkynyl, C₁-C₆alkoxy, C₃-C₇cycloalkyl(C₀-C₄alkyl), C₁-C₆haloalkyl, C₁-C₆haloalkoxy, and mono- and di-(C₁-C₄alkyl)amino;
R₃ₐ and R_{3b} are independently hydrogen, hydroxy, halogen, cyano, C₁-C₆alkyl, C₂-C₆alkenyl, C₂-C₆alkynyl, C₁-C₆alkoxy, C₃-C₇cycloalkyl(C₀-C₄alkyl), C₁-C₆haloalkyl or C₁-C₆haloalkoxy; or R₃ₐ and R_{3b} are taken together to form an oxo group;
R₄ represents from 0 to 3 substituents independently chosen from hydroxy, halogen, amino, nitro, cyano, C₁-C₆alkyl, C₂-C₆alkenyl, C₂-C₆alkynyl, C₁-C₆alkoxy, C₃-C₇cycloalkyl(C₁-C₄alkyl), C₁-C₆haloalkyl, and C₁-C₆haloalkoxy;
Rₐ is independently chosen at each occurrence from:
(i) hydroxy, halogen, amino, aminocarbonyl, cyano, nitro, and —COOH; and
(ii) C₁-C₈alkyl, C₂-C₈alkenyl, C₂-C₈alkynyl, C₁-C₈alkoxy, C₃-C₇cycloalkyl(C₀-C₄alkyl), C₁-C₈haloalkyl, C₁-C₈haloalkoxy, C₁-C₈alkanoyl, C₃-C₈alkanone, C₂-C₈alkoxycarbonyl, C₂-C₈alkanoyloxy, C₁-C₈alkylthio, C₂-C₈alkyl ether, phenylC₀-C₄alkyl, phenylC₀-C₄alkoxy, mono- and di-(C₁-C₆alkyl)aminoC₀-C₆alkyl, and (4- to 7-membered heterocycle)C₀-C₄alkyl; each of which is substituted with from 0 to 3 substituents independently chosen from hydroxy, halogen, amino, cyano, C₁-C₄alkyl, C₁-C₄alkoxy, C₁-C₄haloalkyl, C₁-C₄haloalkoxy, and mono- and di-(C₁-C₄alkyl)amino.

54. A method according to claim 53, wherein the disease or disorder is an eating disorder, sexual disorder, diabetes, heart disease or stroke.

55. A method according to claim 53, wherein the compound or salt is administered orally.

56. A method according to claim 53, wherein the compound or salt is administered intranasally, intravenously or topically.

57. A method according to claim 53, wherein the patient is a human.

58. A method according to claim 53, wherein the patient is a dog or a cat.

59. A method for treating obesity, comprising administering to a patient in need of such treatment a therapeutically effective amount of a compound of claim 1.

60. A method according to claim 59, wherein the compound or salt is administered orally.

61. A method according to claim 59, wherein the patient is a human.

62. A method according to claim 59, wherein the patient is a dog or a cat.

63-66. (canceled)

67. A compound or pharmaceutically acceptable salt thereof wherein the compound is:
(3-Benzyl-phenyl)-(6,7-dimethoxy-3,4-dihydro-1H-isoquinolin-2-yl)-methanone;
(6,7-Dimethoxy-3,4-dihydro-1H-isoquinolin-2-yl)-[6-(2-ethyl-phenoxy)-pyridin-2-yl]-methanone;
(6,7-Dimethoxy-3,4-dihydro-1H-isoquinolin-2-yl)-[6-(3-ethyl-phenoxy)-pyridin-2-yl]-methanone;
2-(3-Benzyl-benzyl)-6,7-dimethoxy-1,2,3,4-tetrahydroisoquinoline;
2-[1-(3-Benzyloxy-phenyl)-ethyl]-1,2,3,4-tetrahydroisoquinoline;
2-[1-(3-Benzyloxy-phenyl)-ethyl]-6,7-dimethoxy-1,2,3,4-tetrahydroisoquinoline;
2-[1-(3-Phenoxy-phenyl)-ethyl]-1,2,3,4-tetrahydroisoquinoline;
2-[1-(4-Benzyloxy-3,5-dimethyl-phenyl)-ethyl]-1,2,3,4-tetrahydroisoquinoline;
2-[1-(4-Benzyloxy-3,5-dimethyl-phenyl)-ethyl]-6,7-dimethoxy-1,2,3,4-tetrahydroisoquinoline;
2-[1-(4-Benzyloxy-phenyl)-ethyl]-1,2,3,4-tetrahydroisoquinoline;
2-[1-(4-Benzyloxy-phenyl)-ethyl]-6,7-dimethoxy-1,2,3,4-tetrahydroisoquinoline;
2-[1-(4-Phenoxy-phenyl)-ethyl]-1,2,3,4-tetrahydroisoquinoline;
2-[2-(4-Isopropyl-phenoxy)-pyridin-4-ylmethyl]-6,7-dimethoxy-1,2,3,4-tetrahydroisoquinoline;
2-[2-(4-tert-Butyl-phenoxy)-pyridin-4-ylmethyl]-6,7-dimethoxy-1,2,3,4-tetrahydroisoquinoline;
2-[3-(3,4-Dichloro-phenoxy)-benzyl]-6,7-dimethoxy-1,2,3,4-tetrahydroisoquinoline;
2-[3-(4-Ethoxy-phenoxy)-benzyl]-6,7-dimethoxy-1,2,3,4-tetrahydroisoquinoline;
2-[3-(4-Isopropyl-phenoxy)-benzyl]-6,7-dimethoxy-1,2,3,4-tetrahydroisoquinoline;
2-[3-(4-tert-Butyl-phenoxy)-2-methyl-benzyl]-6,7-dimethoxy-1,2,3,4-tetrahydroisoquinoline;
2-[3-(4-tert-Butyl-phenoxy)-4-methyl-benzyl]-6,7-dimethoxy-1,2,3,4-tetrahydroisoquinoline;
2-[3-(4-tert-Butyl-phenoxy)-benzyl]-6,7-dichloro-1,2,3,4-tetrahydroisoquinoline;
2-[3-(4-tert-Butyl-phenoxy)-benzyl]-6,7-diethoxy-1,2,3,4-tetrahydroisoquinoline;
2-[3-(4-tert-Butyl-phenoxy)-benzyl]-6,7-dimethoxy-3-methyl-1,2,3,4-tetrahydroisoquinoline;
2-[3-(4-tert-Butyl-phenoxy)-benzyl]-6,7-dimethoxy-1,2,3,4-tetrahydroisoquinoline;
2-[3-(4-tert-Butyl-phenoxy)-benzyl]-6,7-dimethoxy-1,1-dimethyl-1,2,3,4-tetrahydroisoquinoline;
2-[3-(4-tert-Butyl-phenoxy)-benzyl]-6,7-dimethoxy-7-methoxy-1,2,3,4-tetrahydroisoquinoline;
2-[3-(4-tert-Butyl-phenoxy)-benzyl]-7-ethoxy-6-methoxy-1,2,3,4-tetrahydroisoquinoline;
[2-(4-tert-Butyl-phenoxy)-pyridin-4-yl]-(6,7-dimethoxy-3,4-dihydro-1H-isoquinolin-2-yl)-methanone;
2-[6-(2-Ethyl-phenoxy)-pyridin-2-ylmethyl]-6,7-dimethoxy-1,2,3,4-tetrahydroisoquinoline;
2-[6-(3-Ethyl-phenoxy)-pyridin-2-ylmethyl]-6,7-dimethoxy-1,2,3,4-tetrahydroisoquinoline;
2-[6-(4-tert-Butyl-phenoxy)-pyridin-2-ylmethyl]-6,7-dimethoxy-1,2,3,4-tetrahydroisoquinoline;
2-{1-[3-(3,4-Dichloro-phenoxy)-phenyl]-ethyl}-1,2,3,4-tetrahydroisoquinoline;
2-{3-[3-(3,4-Dichloro-phenoxy)-phenyl]-ethyl}-6,7-dimethoxy-1,2,3,4-tetrahydroisoquinoline;
2-{1-[3-(4-Chloro-phenoxy)-phenyl]-ethyl}-1,2,3,4-tetrahydroisoquinoline;
2-{1-[3-(4-Chloro-phenoxy)-phenyl]-ethyl}-6,7-dimethoxy-1,2,3,4-tetrahydroisoquinoline;
2-{1-[3-(4-Methoxy-phenoxy)-phenyl]-ethyl}-1,2,3,4-tetrahydroisoquinoline;
2-{1-[3-(4-tert-Butyl-phenoxy)-phenyl]-ethyl}-1,2,3,4-tetrahydroisoquinoline;
2-{1-[3-(4-tert-Butyl-phenoxy)-phenyl]-ethyl}-6,7-dimethoxy-1,2,3,4-tetrahydroisoquinoline;
[3-(4-tert-Butyl-phenoxy)-2-methyl-phenyl]-(6,7-dimethoxy-3,4-dihydro-1H-isoquinolin-2-yl)-methanone;
4-[3-(6,7-Dimethoxy-3,4-dihydro-1H-isoquinolin-2-ylmethyl)-phenoxy]-benzonitrile;
6,7-Dimethoxy-2-(3-p-tolyloxy-benzyl)-1,2,3,4-tetrahydroisoquinoline;
6,7-Dimethoxy-2-[1-(3-phenoxy-phenyl)-ethyl]-1,2,3,4-tetrahydroisoquinoline;
6,7-Dimethoxy-2-[1-(4-phenoxy-phenyl)-ethyl]-1,2,3,4-tetrahydroisoquinoline;
6,7-Dimethoxy-2-[3-(3,4,5-trimethoxy-phenoxy)-benzyl]-1,2,3,4-tetrahydroisoquinoline;
6,7-Dimethoxy-2-[3-(4-methoxy-phenoxy)-benzyl]-1,2,3,4-tetrahydroisoquinoline;
6,7-Dimethoxy-2-[3-(4-phenoxy-phenoxy)-benzyl]-1,2,3,4-tetrahydroisoquinoline;
6,7-Dimethoxy-2-[3-(4-trifluoromethoxy-phenoxy)-benzyl]-1,2,3,4-tetrahydroisoquinoline;
6,7-Dimethoxy-2-[3-(4-trifluoromethyl-phenoxy)-benzyl]-1,2,3,4-tetrahydroisoquinoline;
6,7-Dimethoxy-2-{1-[3-(4-methoxy-phenoxy)-phenyl]-ethyl}-1,2,3,4-tetrahydroisoquinoline;
6-[3-(4-tert-Butyl-phenoxy)-benzyl]-5,6,7,8-tetrahydro-[1,3]-dioxolo[4,5-g]isoquinoline;
7-Ethoxy-2-[2-(4-isopropyl-phenoxy)-pyridin-4-ylmethyl]-6-methoxy-1,2,3,4-tetrahydroisoquinoline;

68-121. (canceled)
